(19) United States
(12) Patent Application Publication
(10) Pub. No.: US 2006/0194259 A1
(43) Pub. Date: Aug. 31, 2006

(54) Method for isolating an allosteric effector of a receptor

(76) Inventors: Jean-Luc Galzi, Strasbourg (FR); Marcel Hibert, Eschau (FR); Jean-Jacques Bourguignon, Hipsheim (FR); Emalina Maillet, Bauneur (FR)

Correspondence Address: YOUNG & THOMPSON, 745 SOUTH 23RD STREET, 2ND FLOOR, ARLINGTON, VA, 22202, US

(21) Appl. No.: 10/518,298

(22) PCT Filed: Jun. 16, 2003
(86) PCT No.: PCT/FR03/01817

§ 371 Date: Feb. 9, 2006

(30) Foreign Application Priority Data
Jun. 17, 2002 (FR) 02/07436

(51) Int. Cl. G01N 33/567; G01N 33/53

(52) U.S. Cl. 435/7.2

(57) ABSTRACT

A method for isolating an allosteric effector of a receptor, by determining the variation of the dissociation kinetics of the complex formed between the receptor and one of its ligands in the presence of the allosteric effector, as compared to the kinetics dissociation, in the absence of the effector, and/or the amplitude of the linkage formed between the receptor and the ligand in the presence of the allosteric effector, as compared to the amplitude in the absence of the effector. The receptor and ligand are being involved in at least one biological response, and the allosteric effector is capable of modulating at least one of the responses. The receptor is marked by at least one fluorescent protein, and the ligand by a molecule capable of absorbing light, or by a fluorescent substance.

A subject of the present invention is a process for detecting an allosteric effector of a receptor.

Numerous medicaments and natural substances take effect by interacting with regulating proteins, called receptors, involved in numerous physiological functions of organisms, and alterations of their functions are the cause of numerous pathologies.

Amongst the molecules which act by binding to receptors, a particular category, called allosteric effectors or modulators, takes effect by binding to sites, present at the surface of the receptors, but topologically distinct from the binding site of the endogenous ligand. Their interaction with the receptor is of a non-competitive nature relative to that of the endogenous ligand. By binding to their regulating sites, the effectors have little or no effect on the responses of the receptor in the absence of endogenous ligand. On the other hand, they modify the binding properties of the competitive endogenous ligands and alter the biological responses caused by said ligand.

The allosteric effectors are particularly useful in the therapeutic field because their action is not “constitutive” but is revealed only when the endogenous ligand stimulates the receptor. In the case where such molecules have been able to be identified and introduced onto the market, the patient's comfort and the selectivity of the effect are always found to be greatly improved, for example in comparison with the effect observed on the administration of an agonist (Pan et al., 2001).

In order to identify new allosteric effectors of receptors, several experimental approaches have now been developed, in particular measurements of the binding of a ligand to its receptor and functional measurements of physiological responses.

Amongst the methods for measuring binding, there is the study of the dissociation kinetics of a radioligand. As described in Kostenis and Mohr (1996), allosteric modulation is most generally detected by an effect of the allosteric effector on the dissociation kinetics of a radioligand. This method has been implemented in order to demonstrate the non-competitive (and therefore allosteric) character of molecules regulating the muscarinic receptors M1 to M5 of acetylcholine (Tucek and Poska, 1995), A1 of adenosine (Bruns & Ferguns, 1990), alpha 2 of adrenaline (Nunnari et al., 1987), or D2 of dopamine (Hoare & Strange, 1995). In these different examples, the authors indiscriminately used either agonists, or antagonists such as radioligands, and defined the positive or negative character of the modulator by its ability to accelerate (positive) or slow down (negative) the dissociation rate of the radioligand used.

The second most widespread approach to identification of an allosteric effector consists of measuring the quantity of radioligand/receptor complex at a subsaturating concentration of radioligand. Under these conditions, the fact that a third molecule causes an increase in the quantity of radioligand/receptor complex is interpreted in terms of potentialization of binding by an allosteric effect. The work of Bruns and Ferguns, cited above, illustrates this point when an agonist radioligand is used, whereas the article of Jakubik et al. (1997) uses a radioactive antagonist.

The third experimental approach consists of establishing the saturation curve for a radoligand in the presence and in the absence of allosteric effector. In this case, which is illustrated in the article of Massot et al. (1996) for serotonin receptor 5HT1B, the 5HT-moduline effector reduced the quantity of receptor sites without affecting the apparent affinity determined for the radioligand used.

The measurements of the biological response caused by a receptor ligand include either its activation (by the agonists) or its inhibition (by the antagonists). These measurements generally estimate a variation in amplitude of the responses which are either potentialized by the positive effectors or inhibited by the negative effectors of the response, and include:

the recording of ionic currents by electrophysiological methods (Krause et al., 1997; Galzi et al., 1996) or by optical methods using specific ion probes (Birdsall et al., 1999),

the measurement of the production of secondary messengers such as cAMP (Bruns & Ferguns, 1990) or inositol phosphates (Waugh et al., 1999),

the binding of GTP to the G protein associated with the receptor (Birdsall et al., 1999; Lazareno & Birdsall, 1995; Hoare et al., 2000) or the hydrolysis of GTP by the G protein associated with the receptor (Birdsall et al., 1999).

The technique of fluorescence energy transfer (FRET) in combination with the use of a receptor rendered fluorescent and of one of its fluorescent ligands makes it possible to measure, as described in the international Application WO 98/55873, non-covalent interactions between said receptor and said ligand.

The receptor can be rendered fluorescent thanks to a fluorescent protein, for example a protein originating from the Aequorea victoria jellyfish the corresponding gene of which has been sequenced, cloned (Prasher et al., 1992) and optimized for a good expression in the higher eukaryotes (Cormack et al., 1996). It can be fused with the gene coding for another protein, in particular a receptor coupled to a G protein (Galzi & Alix, 1997; Weill et al., 1999; Vollmer et al., 1999; Valenzuela et al., 2001) in order to allow the expression of a fluorescent receptor protein. The combination of the fluorescent receptor protein with one of its fluorescent ligands allows the formation of a non-covalent receptor-ligand complex which is detected by fluorescence energy transfer (Galzi & Alix, 1997; Vollmer et al., 1999; Valenzuela et al., 2001) and the formation and dissociation kinetics of which can be measured (Palanché et al., 2001; Valenzuela et al., 2001).

One of the purposes of the invention is to provide a process which is simple to implement, quick, sensitive, making it possible to carry out quantitative measurements of the interactions between a receptor and a ligand marker of the site of the endogenous ligand, and allowing detection of the presence of any allosteric effector.

Another purpose of the invention is to allow measurements in real time of the interactions of a ligand marker of the site of the endogenous ligand with its receptor, allowing detection of the presence of any allosteric effector.

Another purpose of the invention is to allow time-resolved measurement of the association and dissociation kinetics of a fluorescent ligand, which is a marker of the site of the endogenous ligand, with its receptor, and to detect the presence of any allosteric effector.

Another purpose of the invention is to allow the detection of the interaction between a receptor and its endogenous ligand by fluorescence energy transfer in ranges of endogenous fluorescent ligand concentrations considerably broader (from nanomolar to micromolar) than when a radioligand is used (nanomolar field). Thus, the present invention allows detection by energy transfer of a larger number of conformational states of the receptor, which greatly facilitates the identification and interpretation of the effects of supposed modulating agents.

One of the purposes of the invention is to provide an experimental measurement of the association and dissociation rate constants of a fluorescent ligand, which is a marker of the site of the endogenous ligand, with its receptor, by allowing the detection of the presence of any allosteric effector.

A purpose of the invention is also to provide a process for detecting an allosteric effector of a receptor, which can be applied generally to numerous receptor proteins and their ligands.

A purpose of the invention is also to provide a process for detecting an allosteric effector of a receptor, which can be automated, requiring the purification neither of the receptor, nor of the ligand.

A purpose of the invention is also to provide a process for detecting an allosteric effector of a receptor, non-polluting since it uses no radioactivity, economic since it uses visible light and can be implemented with existing equipment.

The present invention relates to a process for detecting an allosteric effector of a receptor, by determination of the variation:

in the dissociation and/or association kinetics of the complex formed between the abovementioned receptor and one of its ligands in the presence of said allosteric effector, relative to the dissociation and/or association kinetics of the complex formed between said receptor and said ligand, in the absence of said effector,

and/or in the amplitude of the bond formed between the abovementioned receptor and one of its ligands in the presence of said allosteric effector, relative to the amplitude of the bond formed between said receptor and said ligand, in the absence of said effector,

said receptor and said ligand being involved in at least one biological response under appropriate physiological conditions, and the allosteric effector being capable of modulating at least one of the responses,

said receptor being marked by a fluorescent protein chosen from the fluorescent proteins originating or derived from autofluorescent proteins of cnidaria, the molecular extinction coefficient of which is greater than approximately 14000 M⁻¹·cm⁻¹ and the fluorescence quantum efficiency of which is greater than approximately 0.38,

said ligand being marked by a marker constituted:
either by a molecule capable of absorbing the light emitted by the fluorescent protein,
or by a fluorescent substance,

said determinations of the variation in dissociation and/or association kinetics and variation in amplitude being carried out by fluorescence energy transfer:
between the abovementioned fluorescent protein and the abovementioned fluorescent substance, the fluorescent substance being such that it is excitable at the emission wavelength of the abovementioned fluorescent protein, or it emits at the excitation wavelength of the abovementioned fluorescent protein, or
between the abovementioned fluorescent protein and the abovementioned molecule capable of absorbing the light emitted by the fluorescent protein.

The present invention relates to a process for detecting an allosteric effector of a receptor, by determination of the variation:

in the dissociation and/or association kinetics of the complex formed between the abovementioned receptor and one of its ligands in the presence of said allosteric effector, relative to the dissociation and/or association kinetics of the complex formed between said receptor and said ligand, in the absence of said effector,

and/or in the amplitude of the bond formed between the abovementioned receptor and one of its ligands in the presence of said allosteric effector, relative to the amplitude of the bond formed between said receptor and said ligand, in the absence of said effector, provided that when the variation in the abovementioned amplitude is negative, the existence of the variation in the abovementioned kinetics is also detected,

said receptor and said ligand being involved in at least one biological response under appropriate physiological conditions, and the allosteric effector being capable of modulating at least one of the responses,

said receptor being marked by a fluorescent protein chosen from the fluorescent proteins obtained or derived from autofluorescent proteins of cnidaria, the molecular extinction coefficient of which is greater than approximately 14000 M⁻¹·cm⁻¹ and the fluorescence quantum efficiency is greater than approximately 0.38,

said ligand being marked by a marker constituted:
either by a molecule capable of absorbing the light emitted by the fluorescent protein,
or by a fluorescent substance,

said determinations of variation in dissociation and/or association kinetics and of variation in amplitude being carried out by fluorescence energy transfer:
between the abovementioned fluorescent protein and the abovementioned fluorescent substance, the fluorescent substance being such that either it is excitable at the emission wavelength of the abovementioned fluorescent protein, or it emits at the excitation wavelength of the abovementioned fluorescent protein, or
between the abovementioned fluorescent protein and the abovementioned molecule capable of absorbing the light emitted by the fluorescent protein.

The invention responds to the technical problem posed by the determination of the competitive or non-competitive character of the interaction between a receptor and a pharmacological agent supposed to act as effector of a receptor or as competitor of the endogenous ligand of a receptor, by measurements of association and dissociation kinetics. Put simply, the allosteric effector of a receptor alters the association or dissociation kinetics of a receptor whereas an agent competing with the endogenous ligand has no effect on these kinetics.

By “ligand, which is a marker of the site of the endogenous ligand” is meant a ligand which binds to the site of the endogenous ligand by opposition to any allosteric effector, which binds to a site distinct from that of the endogenous ligand. The marker of the site of the endogenous ligand interacts in a competitive manner with the endogenous ligand (mutually exclusive bond), whereas the allosteric effector interacts in non-competitive manner with the endogenous ligand.

By “allosteric effector” or “allosteric modulator” is meant a molecule or a ligand or a biologically active substance which modulates the binding properties of the ligand as well as the functional properties of the receptor, without entering into competition with said ligand.

By definition, it is recalled that by ligand is meant any molecule capable of binding to a receptor in a non-covalent manner by binding to the same site as the endogenous ligand, and that a ligand can be an agonist or an antagonist, an agonist being capable, by binding, of triggering a biological response and an antagonist not triggering a biological response and being capable of blocking the effect of the agonist.

An allosteric effector is also a ligand of the receptor but the latter binds to the receptor on another binding site. In the present context, the term “allosteric effector” or “allosteric modulator” is used and not “ligand”.

Allosteric modulation is the mechanism whereby a molecule, called an allosteric effector or allosteric modulator, increases or reduces the response of a receptor activated by an agonist. The allosteric effector interacts with the receptor at the level of a binding site which is distinct from that at which the competitive ligand binds onto the receptor. The fact that an allosteric modulation is possible resides in:

1) the existence of different conformations or states of the receptor. Each conformation is endowed with distinct pharmacological and functional properties. Thus biologically active and inactive conformations exist. Each conformation binds its ligands with different affinities. For example, an agonist, like a positive allosteric effector, exhibits a greater affinity for the biologically active conformation(s) of the receptor, whereas an antagonist, like a negative allosteric effector, has a greater affinity for the inactive conformation(s) of the receptor;

The pharmacological properties of a conformation are the ability to bind ligands and modulators with a defined affinity. Each conformation binds different ligands with an intrinsic affinity specific to each ligand. All of the ligands binding to a conformation with a specific affinity constitute what is called the pharmacological profile. The pharmacological properties of a conformation are similar to its pharmacological profile.

The functional properties of a conformation of a receptor correspond to its ability or inability to stimulate a biological response. A distinction is made between several functional properties of a receptor:

the quiescent state, by definition non-active, as not coupled to a response, but activable, i.e., capable being interconverted into an active conformation or coupled to a response;

the active state(s), responsible for a biological response; in the case where several active states exists, at least one of the properties of the response is different (for example, calcium response versus cAMP, or then the same response but different sensitivity to the agonists, or also duration of different responses etc.); on the other hand, each active state can give rise to one or more responses, distinct in nature or from a quantitative point of view; moreover, a receptor can adopt several conformations corresponding to several active states with which responses of different types are associated;

the desensitized state(s) which are non-active and non-activable;

2) the existence, on the proteins, of multiple sites which allow the simultaneous interaction of several molecules with a single receptor; the competitive ligands interact with a common site (in mutually exclusive manner) which is itself distinct from the interaction site(s) of the allosteric effectors or modulators;

3) the conformational transitions of the receptor involve all of the receptor molecule and are not restricted to the region of the molecule which binds the ligands; they affect all the tertiary, or even quaternary, structure of the protein, and are discrete; thus, during the conformational transition from the quiescent state to the active state of the receptor, all of the binding sites as well as all of the “biologically active” site see their structure change in a concerted manner.

These different aspects of the functional regulation of a protein are illustrated in FIG. 1 (Monod et al., 1965; Galzi et al., 1996; Rubin and Changeux, 1966). The description of the operating mode of a receptor being able to exist in two conformations (quiescent state and active state) is illustrated in FIG. 3 for a receptor. An equivalent version of this diagram for a receptor coupled to the G proteins being able to activate a Gs protein, itself responsible for the activation of an adenylate cyclase, is described in Tucek and Proska (1995) and in Hall (2000).

In these diagrams, the protein, in particular the receptor, exists in two conformations which are in spontaneous equilibrium with one another. This equilibrium is described by the constant L₀ the value of which is given by the fractional concentration ratio [R]/[A], R being the quiescent state and A the active state. The ligands bind to the R state and to the A state with affinities described by the dissociation constants K_{R} and K_{A}, respectively. If the ratio c=K_{A}/K_{R} is <1, i.e. the affinity of the ligand is better for A than for R, said ligand behaves as an agonist of the receptor and the fractional concentration of receptor in the A state is described by the product L₀c which is itself smaller than L₀. Conversely, if the affinity ratio c is >1, the ligand preferentially binds to the R state and has a tendency to behave as an antagonist. The product L₀c is greater than L₀. The allosteric effectors, by binding to sites distinct from that of the competitive ligands, behave in the same manner as the competitive ligands in that they modify the value of the equilibrium constant between the R and A states. The mathematical formalism of the effect of the allosteric effectors is given in Rubin and Changeux (1966). The presence of an allosteric effector modifies the value of the constant L₀ to L′₀ according to L′₀=L [(1+βd)/(1+β)]ⁿ with β=K_{A}/K_{R} and d=F/K_{A}, F corresponding to the allosteric effector concentration. The addition of an agonist or antagonist then leads to the change in the fractional concentrations of the states of the receptor to L′₀c.

The following definitions are recalled:

By “receptor” is meant any molecule of a proteic nature capable of being involved in non-covalent interaction with a pharmacological agent. Preferentially, in the invention, a neurotransmitter, hormone, growth factor etc. receptor is used, capable of producing, after interaction with a pharmacological ligand, a signal transduction response measurable in vivo and in vitro.

By “competitive interaction” is meant an interaction of two molecules with a receptor taking place at the level of a single site, and a ligand is called “competitive” when it interferes with the binding of another ligand in a steric manner.

In parallel, by “non-competitive interaction” is meant an interaction of two molecules with a receptor taking place at the level of topologically distinct sites, and a ligand is called “non-competitive” when it interferes with the binding of another ligand, the two ligands interacting with topologically distinct sites of the receptor.

It is also recalled that the “association” in the expression “association of the complex” is the action whereby a ligand binds to a receptor protein.

If the receptor protein can adopt several conformations or states, it is sometimes possible to discern the binding of a ligand to the different conformations by differences in association kinetics.

By “association kinetics” is meant the time course of an association reaction. The kinetics can be either monoexponential, or multiexponential. In the case where they are multiexponential, they break down into a sum of monoexponential relaxations each characterized by an association rate and an amplitude.

The “association rate” is measured by means of the rate constant of the association reaction obtained by adjustment of an experimental curve using a monoexponential expression of the form y=λexp (−kₐₚₚ×T) where λ is the amplitude, kₐₚₚ is the apparent rate constant of the reaction and T is time. In the case where the association reflects several events of simultaneous binding, a multiexponential expression of type y=λ₁ exp (−k1ₐₚₚ×T)+λ₂ exp (−k2ₐₚₚ×T)+ . . . is used.

It is recalled that the mathematical expression of the apparent rate constant (kₐₚₚ) depends on the linear or non-linear nature of the variation in kₐₚₚ with the concentration of ligand [L]. Thus, if kₐₚₚ varies in a linear manner with [L], the reaction is bimolecular of the R+L [symbol] RL type with k₁ for the formation and k₋₁ for the dissociation of the complex. kₐₚₚ is then equal to k₁×[L]+k₋₁. If the relation is non-linear, the following reaction diagram is applied: R+L [symbol] RL [symbol] R′L where K_{D} is the dissociation constant of the complex RL, k₂ is the RL→R′L interconversion rate and k₋₂ is the conversion rate constant R′L→RL, kₐₚₚ is then equal to k₂×([L]/([L]+K_{D}))+k₋₂.

It is also recalled that the “dissociation” in the expression “dissociation of the complex” is the action whereby a ligand leaves a receptor site. Dissociation can be obtained in at least two ways: a) by strongly diluting the receptor-ligand mixture in order to favour dissociation relative to association or 2) by adding a strong excess of a competitive ligand which in a favoured manner occupies a site left vacant by the ligand which has been dissociated.

If the receptor protein can adopt several conformations or states, it is sometimes possible to discern the dissociation between a ligand and its receptor having different conformations, by differences in dissociation kinetics between the different conformations.

By dissociation kinetics is meant the time course of a dissociation reaction. The kinetics can be either monoexponential, or multiexponential. In the case where they are multiexponential, they break down into a sum of monoexponential relaxations characterized by a dissociation rate and an amplitude.

The “dissociation rate” is measured by means of the rate constant of the dissociation reaction obtained by adjustment of an experimental curve using an exponential expression of the form y=λ exp(−k×T) where λ is the amplitude, k is the intrinsic rate constant of the reaction (k=k₋₁) and T is time. In the case where the dissociation reflects several simultaneous dissociation events, a multiexponential expression of type y=λ₁ exp (−k₁×T)+λ₂ exp (−k₂×T)+ . . . is used.

It is recalled that the expression “binding amplitude” designates the amplitude of the signal recorded, which is itself proportional to the level occupation of the receptor sites. At a saturating concentration of ligand, the binding amplitude is constant. Below this, it evolves in a non-linear manner, and according to the law of mass action. It can be described by Hill's empirical equation: RL=R₀/(1+K_{D}/L)ⁿ where R₀ is the total quantity of receptor sites present in the test, K_{D} is the dissociation constant, L is the concentration of the ligand, and where n is the proportionality coefficient also called Hill's coefficient.

By “biological response” is meant any variation in metabolism of cells, tissues or organisms. For cells, for example, it is possible to determine variations in pH, ionic concentration, formation of metabolites such as GTP or cAMP, gene expression, cell morphology (measurement of the percentage of cells which have changed form), cell proliferation, inter alia. Examples are given below.

By “appropriate physiological conditions”, is meant all conditions of pH, concentrations and ionic composition, nutritive complements as close as possible to those which are encountered in the whole organism. These conditions are chosen such that the experiment carried out is conducted under conditions as close as possible to those that could be obtained by carrying out the measurement in the whole organism.

The expression “capable of modulating at least one of the responses” signifies that in a set of measurable responses which can be stimulated by a receptor, at least one must be modified by the effector. The modification or modulation can affect the delay in establishment of the response, its frequency, amplitude, duration, extinction rate, as well as its sensitivity to the agonist.

As regards the signal transduction response for the receptors coupled to the G proteins, the general test consists of determining the activation of the G protein by measurement of the binding of GTP (Befort et al., 1996). Other more specific measurements for example involve determinations of intracellular concentrations of cAMP, inositol phosphates, calcium, measuring activation of gene transcription or oncogenic activity, depending on the type of coupling specific to the receptor considered.

For the receptor-channels, the most direct measurements are determinations of ionic currents (Hille, 1992). Other measurements can, for example, involve determining gene transcription or activations of enzymes.

For the growth-factor receptors, the general tests are those of proliferation, differentiation or cell survival, frequently also phosphorylation tests of specific substrates (Honneger et al., 1988) of each receptor and location by specific antibodies of phosphoamino acids.

For the nuclear receptors, the signal transduction tests are those of gene transcription in which reporter genes, for example “chromogenic”, are placed under the control of specific promoters of the transduction routes of the receptor studied (Ko et al., 2002).

Thus, for the cAMP accumulation responses, various protocols can be used, in particular a radioimmunological measurement as described in Palanché et al. (2001) or Hausdorff et al. (1990).

The binding of GTP to the G protein can be recorded for example as described in Fawzi et al. (2001) or Vuong and Chabre (1990).

Cell proliferation is for example analyzed according to the protocol described in Burstein and al. (1997).

The regulation of the gene expression can for example be studied according to the protocol described in Baulmann et al. (2000).

The activation of protein kinases can for example be studied as described in Vollmer et al. (1999) or Yuan et al. (2000).

The variation in pH can be measured (Nicolini et al., 1995).

An “autofluorescent protein” is a natural or synthetic protein in which the chromophore is formed by an autocatalytic reaction between amino acids of the protein without requiring the addition of a prosthetic group (chromophore), and the fluorescence properties of which are intrinsic to the monomer.

The expression “fluorescence energy transfer” corresponds to a physical process, dependent on distance, by which energy is transmitted in a non-radiative manner from an excited chromophore, the donor, to another chromophore, the acceptor, by dipole-dipole interaction (Förster, 1951; Wu and Brand, 1994; Clegg, 1995). The energy transfer can be observed either by a reduction in the amplitude of the donor emission, or by an increase in the amplitude of the acceptor excitation and emission. If the acceptor is not fluorescent, but has an excitation spectrum at least partly covering the donor emission spectrum, the energy transfer can be detected in the form of a reduction in amplitude of the donor emission.

In the case of the application of energy transfer to biological samples in non-covalent interaction, the transfer signal cannot persist if the experimental conditions do not allow the interaction between the fluorescent ligand and the fluorescent receptor. Similarly, if one of the two interacting partners does not carry an appropriate chromophore, any variations in fluorescence observed for the other partner cannot be attributed to an energy transfer process.

The terms “change” or “variation in fluorescence”, defined in the context of energy transfer, refer to any modifications of 1) the amplitude of the fluorescence signal of the acceptor, 2) the amplitude of the excitation spectrum or 3) the amplitude of the donor emission signal. The variations or changes in fluorescence should not be observed if one of the two partners does not carry an appropriate chromophore or fluorophore (see below) or if the interaction between the fluorescent partners is inhibited, for example by an excess of a competing agent.

More precisely, the fluorescence energy transfer reaction requires two groups, one called the donor, which must be fluorescent, and the other called the acceptor, either fluorescent, or dye. This reaction is produced when two conditions are met:

1) the absorption spectrum of the acceptor and the emission spectrum of the donor must cover each other, at least partly; the covering is calculated from experimental data and an equation giving a value in cm³M⁻¹ (Lakey et al., 1991);

2) the donor and the acceptor must be spatially close (from 10 to 100 angstroms) in order for the energy transfer to be able to take place.

The first condition has as a consequence the fact that the excitation of the donor then leads in a concomitant manner to a reduction in the amplitude of the donor emission and the appearance of an acceptor emission signal. This makes it possible to detect the interactions between the donor and the acceptor and/or to measure their distance.

The expression “spatially close” signifies that the distance between the donor and the acceptor is less than 2 Ro, Ro representing Forster's radius (op.cit.) (Lakey et al., 1991).

According to an advantageous embodiment of the invention, when the variation in the amplitude of the bond formed between the abovementioned receptor and one of its ligands in the presence of said allosteric effector, relative to the amplitude of the bond formed between said receptor and said ligand, in the absence of said effector, is negative, the existence should be detected of a variation in the dissociation and/or association kinetics of the complex formed between the abovementioned receptor and one of its ligands in the presence of said allosteric effector, relative to the dissociation and/or association kinetics of the complex formed between said receptor and said ligand, in the absence of said effector, and advantageously this variation in kinetics should be quantified, in order to distinguish an allosteric effector from a competitive agent.

The present invention relates to a process for detecting an allosteric effector of a receptor, by determination of the variation:

in the dissociation and/or association kinetics of the complex formed between the abovementioned receptor and one of its ligands in the presence of said allosteric effector, relative to the dissociation and/or association kinetics of the complex formed between said receptor and said ligand, in the absence of said effector,

and/or in the amplitude of the bond formed between the abovementioned receptor and one of its ligands in the presence of said allosteric effector, relative to the amplitude of the bond formed between said receptor and said ligand, in the absence of said effector,

said receptor and said ligand being involved in at least one biological response under appropriate physiological conditions, and the allosteric effector being capable of modulating at least one of the responses,

said receptor being marked by a fluorescent protein chosen from:
green fluorescent protein (GFP or EGFP), cyan fluorescent protein (CFP or ECFP), yellow fluorescent protein (YFP or EYFP) or GFPUV, or,
variants derived from GFP, CFP, YFP or GFPUV, by addition, deletion or substitution of one or more amino acids, provided that these variants preserve the property of fluorescence,
or fragments of the GFP, CFP, YFP or GFPUV, or fragments of the abovementioned variants, provided that these fragments preserve the property of fluorescence,

said ligand being marked by a marker constituted:
either by a molecule capable of absorbing the light emitted by the fluorescent protein,
or by a fluorescent substance,

said determinations of variation in dissociation and/or association kinetics and variation in amplitude being carried out by fluorescence energy transfer:
between the fluorescent protein as defined above and the abovementioned fluorescent substance, the fluorescent substance being such that either it is excitable at the emission wavelength of the fluorescent protein, or it emits at the excitation wavelength of the fluorescent protein, or
between the fluorescent protein as defined above and the abovementioned molecule capable of absorbing the light emitted by the fluorescent protein.

According to a preferred embodiment of the invention, when the variation in the abovementioned amplitude of the bond formed between the abovementioned receptor and one of its ligands in the presence of said allosteric effector, relative to the amplitude of the bond formed between said receptor and said ligand, in the absence of said effector, is negative, the existence of the variation in the abovementioned kinetics is also detected.

The receptor is marked by genetic route by a fluorescent protein chosen from:

green fluorescent protein (GFP) (Ward et al., 1980; Chalfie, 1995), or EGFP (Heim & Tsien, 1996; Miyawaki et al., 1997),

cyan fluorescent protein (CFP or ECFP) (Heim & Tsien, 1996; Miyawaki et al., 1997),

yellow fluorescent protein (YFP or EYFP) (Cormack et al., 1995; Heim, Cubitt and Tsien, 1995; Ehrig et al., 1995) (Miyawaki et al., 1997),

GFPUV (Crameri et al., 1996; Ehrig et al., 1995),

or their mutants in which the codons are optimized for expression in human, bacterial or plant cells,

or their mutants having higher or lower excitation or emission wavelengths than those associated with the proteins defined above, provided that their molecular extinction coefficient is greater than approximately 14000 M⁻¹cm⁻¹ and their fluorescence quantum efficiency is greater than approximately 0.38.

The expression “optimized codons” indicates the substitution of codons of the wild-type protein by their preferred homologues of the host organism, without a change in code therefore without a change in proteic sequence.

The wild-type (WT) GFP with an excitation and emission wavelength of 395/470-509 is described in Ward et al. (1980) and Chalfie (1995).

The UV GFP having the following mutations: F99S, M153T, V163A with an excitation and emission wavelength of 395-510 respectively is described in Crameri et al. (1996), or with the mutation T203I and the excitation and emission wavelength of 400-512 respectively is described in Ehrig et al. (1995).

EGFP has the following mutations:

| | | |
|---|---|---|
| F64L | S65T | H231L |

EYFP has the following mutations:

| | | | |
|---|---|---|---|
| S65G | V68L | S72A | T203Y |

ECFP has the following mutations:

| | | | |
|---|---|---|---|
| F64L | S65T | Y66W | N146I |
| M153T | V163A | N212K | |

The different mutants of GFP can moreover be optimized (by the introduction of silent mutations optimizing the use of codons specific to each species) for expression in the following cells:

human (Haas et al., 1996; Yan et al., 1996; Zolotukhin et al., 1996)

bacterial (Crameri et al., 1996; Cormack et al., 1996, for Escherichia coli),

plant (Reichel et al., 1996).

The term GFP indicates a protein which once expressed in cells emits a fluorescence. GFPs having substitutions, additions or deletions of amino acids influencing either the fluorescence properties, or the level of expression of GFP are called GFP mutants.

The chief characteristics of the fluorescent proteins advantageously used in the process of the invention are given below:

| | maximal λ- | | extinction | quantum |
|---|---|---|---|---|
| Protein | excitation | λ-emission | coefficient | efficiency |
| EYFP | 514 | 527 | 36500 | 0.63 |
| ECFP | 432 | 480 | 18000 | 0.67 |
| GFPUV | 395 | 509 | 21000 | 0.77 |
| EGFP | 489 | 511 | 39000 | 0.66 |

The autofluorescent protein BFP is preferably excluded as it does not correspond to the conditions defined here for autofluorescent proteins of cnidaria, namely molecular extinction coefficient greater than 14000 M⁻¹cm⁻¹ and fluorescence quantum efficiency greater than 0.38.

According to an advantageous embodiment of the invention, the receptor is marked by a fluorescent protein (No. 1) and the ligand is marked

either by a fluorescent substance, the marking being:

either carried out by chemical route, the fluorescent substance then being a chemical compound,

or carried out by recombinant route, the fluorescent substance then being a peptide or a fluorescent protein (No. 2), and being able to be in particular chosen from the fluorescent proteins obtained or derived from autofluorescent proteins of cnidaria, the molecular extinction coefficient of which is greater than approximately 14000 M⁻¹cm⁻¹ and the fluorescence quantum efficiency of which is greater than approximately 0.38, in particular chosen from:

green fluorescent protein (GFP), or

variants derived from GFP, by addition, deletion or substitution of one or more amino acids, provided that these variants preserve the property of fluorescence,

or fragments of GFP, or fragments of the abovementioned variants, provided that these fragments preserve the property of fluorescence,

or by a non-fluorescent substance belonging to the group of acid violets [Acid Violet 5, CAS 10130-48-0; Acid Violet 7, CAS 4321-69-1; Acid Violet 17, CAS 4129-84-4], acid reds [Acid Red 1, CAS 3734-67-6; Acid Red 8, CAS 4787-93-3; Acid Red 37, CAS 6360-07-2; Acid Red 40, CAS 12167-45-2; Acid Red 106, CAS 6844-74-2; Acid Red 114, CAS 6459-94-5], alizarins, aluminon, azocarmine B [CAS 25360-72-9], basic fuschine [Basic Red 9, CAS 569-61-9], Bordeaux R [Acid Red 17, CAS 5858-33-3], Carmine [CAS 1390-65-4].

“CAS” corresponds to Chemical Abstracts.

By marking of a receptor or ligand, is meant:

for the receptor, the fusion of its gene or cDNA, or part of the gene or cDNA, with the gene or cDNA, or part of the gene or cDNA, of GFP;

for the ligand, it can be a chemical coupling between the ligand and a fluorescent group, or fusion of its gene or cDNA, or part of the gene or cDNA, with the gene or cDNA, or part of the gene or cDNA, of GFP.

The invention relates to the use of a fluorescent protein according to the invention in which the receptor and the ligand are marked by genetic route, the fluorescent protein and the fluorescent substance being respectively chosen from the following pairs of compounds:
GFPUV-EYFP
EYFP-GFPUV
ECFP-EYFP
EYFP-ECFP
ECFP-EGFP
EGFP-ECFP
EGFP-EYFP
EYFP-EGFP

and in particular in which the receptor is marked by the protein EYFP or EGFP and the ligand is marked by the protein ECFP, or the receptor is marked by the protein ECFP and the ligand is marked by the protein EYFP or EGFP.

According to an advantageous embodiment of the invention, the fluorescent protein is EGFP and:

either the EGFP is a donor of fluorescence energy and the marker absorbing the light emitted by the EGFP is a fluorescent or non-fluorescent substance, and the marker being chosen from substances, the excitation spectrum of which overlaps the emission spectrum of the EGFP,
and, in the case where the marker is a fluorescent substance, it is chosen from: 4,4-difluoro-4-bora-3a,4a-diaza-s-indacene (Bodipy), eosine, lissamine, erythrosine, tetramethylrhodamine, sulphorhodamine 101 marketed by Molecular Probe under the name Texas Red, and their derivatives allowing on the one hand grafting and, on the other hand, the excitation spectrum of which covers the emission spectrum of the EGFP,
and, in the case where the marker is not a fluorescent substance, it is chosen from the group of acid violets [Acid Violet 5, CAS 10130-48-0; Acid Violet 7, CAS 4321-69-1; Acid Violet 17, CAS 4129-84-4], acid reds [Acid Red 1, CAS 3734-67-6; Acid Red 8, CAS 4787-93-3; Acid Red 37, CAS 6360-07-2; Acid Red 40, CAS 12167-45-2; Acid Red 106, CAS 6844-74-2; Acid Red 114, CAS 6459-94-5], alizarines, aluminon, azocarmine B [CAS 25360-72-9], basic fuschine [Basic Red 9, CAS 569-61-9], Bordeaux R [Acid Red 17, CAS 5858-33-3], Carmine [CAS 1390-65-4],

or the EGFP is an acceptor of fluorescence energy and the fluorescent substance is a donor of fluorescence energy and is chosen from substances, the emission spectrum of which overlaps the excitation spectrum of the EGFP, and in particular from: the coumarines, fluorescamine, 6-(N-methylanilino)naphthalene, (mansyl) and their derivatives allow on the one hand grafting and, on the other hand, the emission spectrum of which covers the excitation spectrum of the EGFP,

According to an advantageous embodiment of the invention, the fluorescent protein is ECFP and:

either the ECFP is a donor of fluorescence energy and the fluorescent substance is an acceptor of energy and is chosen from fluorescein and 7-nitrobenz-2-oxa-1,3-diazole,

or the ECFP is an acceptor of fluorescence energy and the fluorescent substance is a donor of energy and is chosen from pyrene or coumarine or their derivatives allowing on the one hand grafting, and, on the other hand, the emission spectrum of which overlaps the excitation spectrum of the ECFP.

As regards the receptor, it can be chosen from:

membrane receptors coupled to the G protein, in particular in Supplement Trends in Pharmacological Sciences, 1997 (Receptor and ion Channel Nomenclature) and in the databases ensembl.org and GPCRdb,

growth factor receptors, in particular those which are structurally linked to the insulin receptor (Yarden, Y. and Ullrich, A., 1988) or to the γ interferon receptor (Brisco et al., 1996; Ihle, 1995) and those described in the databases ensembl.org and GPCRdb,

the receptor channels, in particular in Supplement Trends in Pharmacological Sciences, 1997 (Receptor and ion Channel Nomenclature) and those described in the databases ensembl.org and GPCRdb,

the intracellular nuclear receptors, in particular those which are structurally linked to the steroids receptor (Mangelsdorf et al., 1995; Wurtz et al., 1996) and those described in the databases ensembl.org and GPCRdb.

According to an advantageous embodiment, the receptor is chosen from the membrane receptors coupled to the G protein.

As defined above, it is recalled that by “receptor” is meant any molecule of proteic nature capable of entering into a non-covalent interaction with a pharmacological agent, and, preferentially, a neurotransmitter, hormone, growth factor etc. receptor, capable of producing, after interaction with a pharmacological ligand, a signal transduction response measurable in vivo and/or in vitro.

By signal transduction response, is meant any response, or response inhibition, measurable in vivo and/or in vitro, resulting from the interaction of a receptor with its specific pharmacological agents and leading to activations or inhibitions of the cell metabolism by an effect on secondary messengers, enzymes, or ionic currents.

As examples of membrane receptors coupled to the G proteins, there can be mentioned the receptors of purines and nucleotides, biogenic amines, peptides and proteins, eicosanoids, lipids and derivatives, exciter amino acids and ions, olfactory molecules as well as orphan receptors (hereafter a fairly exhaustive list).

As examples of growth-factor receptors, there can be mentioned cytokines, epidermal growth factor, insulin, growth factor derived from platelets, transforming growth factor.

As receptor channels, there can be mentioned in particular the receptors of ATP, serotonin, GABA, glycine, acetylcholine, glutamate.

As examples of nuclear receptors, there can be mentioned in particular the receptors of the thyroid hormones, estrogens, glucocorticoids, retinoids.

As ligands of the receptors coupled to the G protein there can be mentioned:

Purines and Nucleotides
Adenosine
cAMP
ATP
UTP
ADP

Biogenic Amines (and linked natural ligands)
5-hydroxytryptamine
Acetylcholine
Dopamine
Adrenaline
Histamine
Melatonin
Noradrenaline
Tyramine/Octopamine
other linked compounds

Peptides
Adrenocorticotrophic hormone (ACTH)
Melanocyte-stimulating hormone (MSH)
Melanocortins
Neurotensin (NT)
Bombesin and neighbouring peptides
Endothelins
Cholecystokinin
Gastrin
Neurokinin B (NKB)
Receptor of the tachykinins
Substance K (NKA)
Substance P(SP)
Neuropeptide Y (NPY)
Thyrotropin releasing factor
Nociceptin
Bradykinin
Angiotensin II
Beta-endorphin
C5a anaphalatoxin
Calcitonin
Chemokines (also called intercrines)
Corticotrophin releasing factor (CRF)
Dynorphin
Endorphin
Formylated peptides
Follitropin (FSH)
Fungal maturation pheromones
Galanin
Gastric inhibitory polypeptide (GIP) receptor
Glucagon peptide analogues (GLPs)
Glucagon
Gonadotropin releasing hormone (GmRH)
Growth hormone releasing hormone (GHRM)
Insect diuretic hormone
Interleukin
Leutropin (LH/HCG)
MET-enkephalin
Opioid peptides
Oxytocin
Parathyroid hormone (PTH) and (PTHrP)
Pituitary adenyl cyclase activating peptides (PACAP)
Secretin
Somatostatin
Thrombin
Thyrotropin (TSH)
Vasoactive intestinal peptide (VIP)
Vasopressin
Vasotocin

Eicosanoids
IP-Prostacyclins
PG-Prostaglandins
TX-Thromboxanes

Retinal-based compounds
11-cis retinal of vertebrates
11-cis retinal of invertebrates

Lipids and lipid-based compounds
Cannabinoids
Anandamide
Lysophosphatidic acid
Platelet-activating factor
Leukotrienes

Exciter amino acids and ions
Calcium ion
Glutamate

Orphan receptors

The present invention relates to a process as defined above, characterized in that the variation:

in the dissociation kinetics of the complex formed between the abovementioned receptor and one of its ligands in the presence of said allosteric effector, relative to the dissociation kinetics of the complex formed between said receptor and said ligand, in the absence of said effector, and/or in the amplitude of the bond formed between the abovementioned receptor and one of its ligands in the presence of said allosteric effector, relative to the amplitude of the bond formed between said receptor and said ligand, in the absence of said effector is determined.

The present invention relates to a process as defined above, characterized in that only the dissociation kinetics of the complex formed between the abovementioned receptor and one of its ligands in the presence of said allosteric effector is determined, relative to the dissociation kinetics of the complex formed between said receptor and said ligand, in the absence of said effector.

In all cases, the measurement of the variation in the kinetics is sufficient to detect an allosteric effector.

The usefulness of observing only the dissociation kinetics resides in the fact that at high (saturating) concentrations of fluorescent ligand, it is possible that no variation in amplitude is detectable as all the binding sites are saturated.

The present invention relates to a process as defined above, characterized in that only the amplitude of the bond formed between the abovementioned receptor and one of its ligands in the presence of said allosteric effector is determined, relative to the amplitude of the bond formed between said receptor and said ligand, in the absence of said effector.

In certain cases, simply the measurement of the amplitude is sufficient to detect an allosteric effector, whereas in others, it must be coupled to measurement of the variation in the kinetics.

According to an advantageous embodiment of the present invention, the process as defined above is characterized in that the ligand is an antagonist.

As defined above, it is recalled that by “antagonist”, is meant any molecule inhibiting the effect of the agonist by binding on the same receptor as the latter.

According to an advantageous embodiment of the present invention, the process as defined above is characterized in that the ligand is an agonist.

As defined above, it is recalled that by “agonist”, is meant any molecule binding to the site of the natural endogenous ligand and capable of activating the biological response.

Any complex biological system can be described by a partial model comprising a number of reduced states sufficient to describe the observed phenomenon. In the case of allosteric modulators or effectors, a partial model comprising two states is used: the quiescent state (R) and the active state (A) of the receptor.

There is thus an R [symbol] A equilibrium.

In this model, the ligands have distinct affinities for the R and A states. In a qualitative manner, it is possible to describe the relative affinities K_{R} and K_{A} for R and A respectively as follows, with c=K_{A}/K_{R}:

| | | | |
|---|---|---|---|
| Affinity for | Quiescent state | Active state | c |
| Agonist | less good | better | c < 1 |
| Competitive | better | less good | c ≧ 1 |
| antagonist | | | |

In this description, an agonist shifts the R [symbol] A equilibrium in favour of A as its affinity is better for A than for R. Conversely, the competitive antagonist shifts the equilibrium towards R. The differences in affinity between the R state and the A state vary from 1 time (c=1) (no difference in affinity) to more than 100 times depending on the ligand (c≧100 or c≦0.01). Thus, for a set of ligands of a target, a range of agonist (c<1) or antagonist (c>1) molecules of variable effectiveness is obtained.

In practice, the association of a ligand with a receptor site is controlled by the diffusion of this ligand in the biological medium. Differences in affinity for a given ligand, and for a given site, result from a different dissociation rate of said ligand for the site in each of the conformations that it can adopt. The same applies to the agonists, the competitive antagonists and the allosteric effectors. As a result of this the allosteric effector, by stabilizing for example a conformation of high affinity for the agonist, will increase the proportion of the receptors in the high affinity conformation, and therefore lead to a reduction in the dissociation rate of the agonist bound to its site. In an experimental dissociation rate measurement, a dissociation curve is obtained as a function of time, the general rate of which is slower if the effector stabilizes states of high affinity for the fluorescent ligand. These curves are correctly analyzed according to a multiexponential model in which the dissociation rates are identical in the presence and in the absence of effector, but the amplitudes of which (and therefore the fractional concentrations of the various conformational states) differ. Thus [Quiescent]/[Active] concentration ratios are obtained which differ when the agonist alone is present and when the agonist and the allosteric effector are both present. In the same manner, the experiment can be carried out with an antagonist: in this case, the effect of the allosteric effectors has the opposite sign to that which is observed for an agonist.

If a dissociation is carried out after association at equilibrium, this difference in affinity between R and A can be revealed by dissociation kinetics having a monoexponential (0.01≦L₀c≦100) or biexponential time course for L₀c>100 or L₀c<0.01. In the case where the kinetics are biexponential, they are described using 2 rate constants and 2 amplitudes, and the ratio of the 2 amplitudes corresponds to the fractional concentration of each of the states. When the dissociation kinetics are biexponential, the sum of the dissociation events of each of the conformational states is recorded.

In the same way as previously, in the case of the effectors, the relative affinities K_{R} and K_{A} for R and A respectively can be described as follows, with c=K_{A}/K_{R}:

| | | | |
|---|---|---|---|
| Affinity for | Quiescent state | Active state | c |
| Positive Effector | less good | better | c < 1 |
| Negative Effector | better | less good | c ≧ 1 |

In this description, a positive effector shifts the R [symbol] A equilibrium in favour of A as its affinity is better for A than for R. Conversely, the negative effector shifts the equilibrium towards R.

Thus, in the case where two states exist, namely an active state and a quiescent state, the present invention relates to a process as defined above, characterized in that the dissociation kinetics of the complex formed between said receptor and one of its ligands (agonist), in the presence of said effector, is slower than the dissociation kinetics of the complex formed between said receptor and said ligand, in the absence of said effector, which means that the allosteric effector is a positive effector. In this preferred embodiment of the invention, the ligand is an agonist: it therefore shifts the R [symbol] A equilibrium towards A (active state of the receptor) for which it has a higher affinity and therefore a slower dissociation rate, whereas the allosteric effector also shifts the equilibrium towards A, which has the effect of increasing the amplitude of the slow dissociation and reducing the amplitude of the rapid dissociation.

In the case where two states exists, namely an active state and a quiescent state, the present invention relates to a process as defined above, characterized in that the dissociation kinetics of the complex formed between said receptor and one of its ligands (agonist), in the presence of said effector, is more rapid than the dissociation kinetics of the complex formed between said receptor and said ligand, in the absence of said effector, which means that the allosteric effector is a negative effector. In this preferred embodiment of the invention, the ligand is an agonist: it therefore shifts the R [symbol] A equilibrium towards A (active state of the receptor) for which it has a higher affinity and therefore a slower dissociation rate, whereas the allosteric effector also shifts the equilibrium towards R, which has the effect of reducing the amplitude of the slow dissociation and increasing the amplitude of the rapid dissociation.

In the case where two states exists, namely an active state and a quiescent state, the present invention relates to a process as defined above, characterized in that the dissociation kinetics of the complex formed between said receptor and one of its ligands, in the presence of said effector, is slower than the dissociation kinetics of the complex formed between said receptor and said ligand, in the absence of said effector, which means that the allosteric effector is a negative effector. In this preferred embodiment of the invention, the ligand is an antagonist: it therefore shifts the R [symbol] A equilibrium towards R (quiescent state of the receptor) for which it has a higher affinity and therefore a slower dissociation rate, whereas the allosteric effector stabilizes the quiescent state, which has the effect of increasing the amplitude of the slow dissociation, whilst reducing the amplitude of the rapid dissociation.

In the case where two states exists, namely an active state and a quiescent state, the present invention relates to a process as defined above, characterized in that the dissociation kinetics of the complex formed between said receptor and one of its ligands, in the presence of said effector, is more rapid than the dissociation kinetics of the complex formed between said receptor and said ligand, in the absence of said effector, which means that the allosteric effector is a positive effector. In this preferred embodiment of the invention, the ligand is an antagonist: it therefore shifts the R [symbol] A equilibrium towards R (quiescent state of the receptor) for which it has a higher affinity and therefore a slower dissociation rate, whereas the allosteric effector stabilizes the active state, which has the effect of reducing the amplitude of the slow dissociation, and increasing the amplitude of the rapid dissociation.

In the case where the receptor can adopt a number of conformations greater than two, the conformations occupied by the fluorescent ligand should be identified in order to determine to which conformational equilibriums the effect of the supposed allosteric agent relates.

Thus, if three conformational states R (quiescent), A (active) and D (desensitized) exist, the agonist can occupy the A and D states whereas the antagonist will preferentially bind to the R state or to the D state. This will result in effects of a nature different from the allosteric agent depending on whether a fluorescent agonist or a fluorescent antagonist is used in the binding measurement experiments. The positive effector (which stabilizes A) reduces the binding of the agonist to the D state. There is an increase in the dissociation rate by the disappearance of a fraction of the receptors in the D state which generally binds the agonist with a greater affinity than the A state.

In the same manner, if two actives states A1 and A2 exist, an effector stabilizing A1 will have the effect of reducing the binding to A2 (and will reduce the responses which are associated with it) whereas it will increase the binding to A1 (and potentialize the responses which are associated with it). In this case, an increase is observed in the dissociation rate of the fluorescent agonist.

The analysis of the association kinetics of the ligand is more complex. It depends in fact on the experimental system analyzed. For certain receptors, and under certain experimental conditions, it is possible to observe multiexponential association kinetics. In these cases, the most rapid kinetics reflect the bimolecular interaction of the ligand (agonist or antagonist) with the receptor, whereas the slower kinetics can reflect conformational interconversions which take place more slowly. If this is observed experimentally, it is then possible to analyze the amplitudes of the slow kinetics and to observe variations in these amplitudes in the presence of an allosteric effector. These variations will reflect the differential stabilization of the various conformational states by the allosteric effector. In other unfavourable cases, the conformational interconversions are kinetically invisible as they are more rapid than the bimolecular association kinetics of the ligand with the receptor. The effect of an allosteric effector on the association rate cannot then be detected experimentally.

Analysis of the responses is a way of monitoring the active state of the receptor. In fact, the active state is defined as a conformational state endowed with the ability to produce the biological response. Analysis of the responses must relate to several parameters:

One of the more simple parameters to be evaluated is the amplitude of the response. The latter is greater in the presence of a positive allosteric effector and lower when the allosteric effector is negative.

The second parameter is that of delay: it is sometimes possible to detect a delay in establishment which is shorter for potentialized responses, and conversely a delay which is longer for inhibited responses, in comparison with a control response. This can in fact be observed when the receptor activates secondary effectors which are themselves responsible for the response. In this case, which is that of the receptors coupled to the G proteins, the transitory accumulation of activated relay proteins (the G protein) or of secondary messengers (inositol triphosphates, cAMP etc.), the appearance of the response can occur with a shorter delay when there is potentialization.

Finally, when several responses can be triggered by a receptor, the comparative analysis of the amplitudes (and/or of the delays) of the various responses can provide indications of the nature of the modulating effect. The present invention also relates to a process as defined above, by determination of:

the variation in the amplitude of the bond formed between the abovementioned receptor and one of its ligands in the presence of said allosteric effector, relative to the amplitude of the bond formed between said receptor and said ligand, in the absence of said effector,

and optionally of the variation in the dissociation kinetics of the complex formed between the abovementioned receptor and one of its ligands in the presence of said allosteric effector, relative to the dissociation kinetics of the complex formed between said receptor and said ligand, in the absence of said effector.

The present invention relates to a process as defined above, characterized in that only the variation in the amplitude of the bond formed between the abovementioned receptor and one of its ligands in the presence of said allosteric effector is determined, relative to the amplitude of the bond formed between said receptor and said ligand, in the absence of said effector, when said variation is positive.

The present invention relates to a process as defined above, characterized in that:

the variation in the amplitude of the bond formed between the abovementioned receptor and one of its ligands in the presence of said allosteric effector, relative to the amplitude of the bond formed between said receptor and said ligand, in the absence of said effector, is determined

and that said variation is negative, which requires the determination of:

the variation in the dissociation kinetics of the complex formed between the abovementioned receptor and one of its ligands in the presence of said allosteric effector, relative to the dissociation kinetics of the complex formed between said receptor and said ligand, in the absence of said effector.

This embodiment thus makes it possible to distinguish an allosteric effector from a competitive agent.

According to an advantageous embodiment of the present invention, the process of the invention is characterized in that said variation in dissociation kinetics is positive or negative, which means that the compound tested is an allosteric effector.

According to an advantageous embodiment of the present invention, the process of the invention is characterized in that said variation in dissociation kinetics is zero, which means that the compound tested is a competitor.

In the case of the receptors coupled to the G proteins complex cases are discerned as several active states of a receptor can exist (Palanché et al., 2001; Lefkowitz, 1998). In this case, the different active states are provided with distinct functional properties, i.e. they regulate responses which can differ from one another. The allosteric effector can then have a behaviour discriminating between the active states. Thus, by favouring the stabilization of one active state taken from several, the effector can behave as positive modulator of a given response whilst behaving as a negative modulator of the responses associated with the other active states. The positive and/or negative character is then defined relative to a given biological response.

The effector can modulate an unknown response of the receptor. Thus, in the case where no effect on a known response on the receptor is observed, the present invention makes it possible to research other responses of the receptor.

The present invention also relates to products corresponding to one of the following formulae:

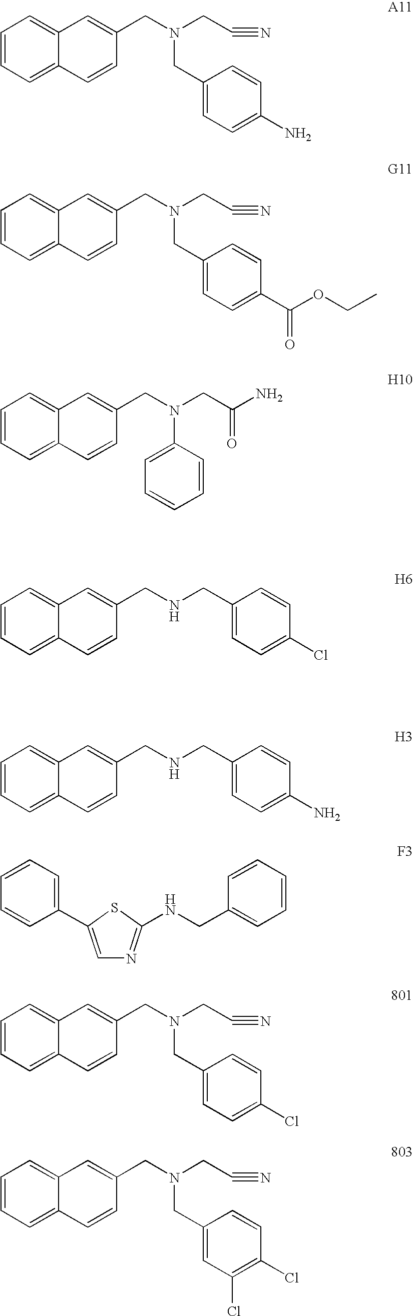

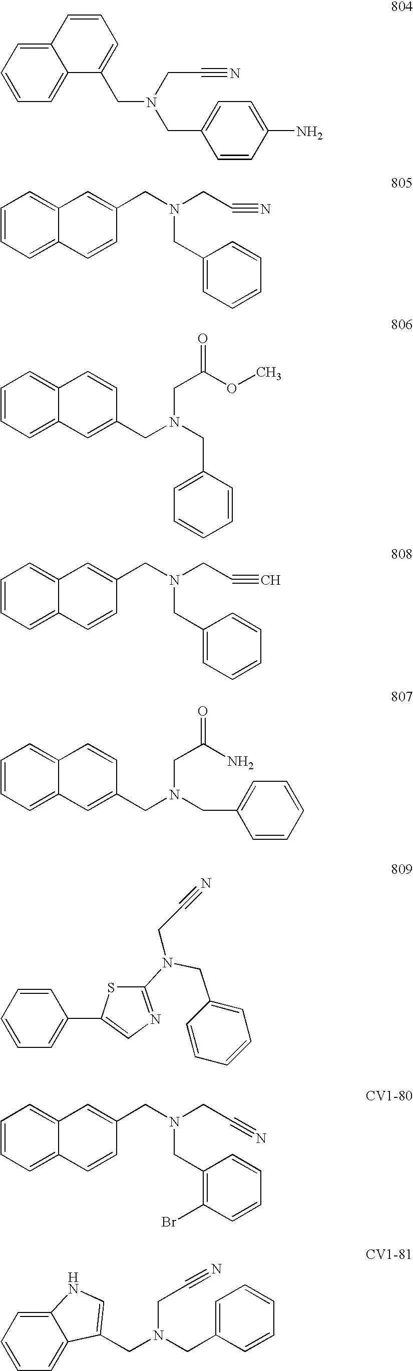

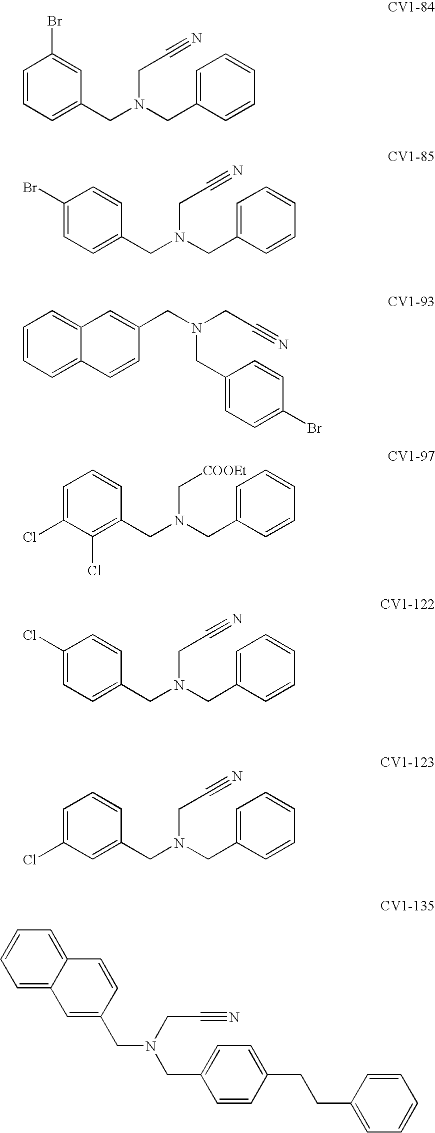

said products being compounds such as detected by the process indicated above.

The products of formula A11, G11, H10, H6, H3, F3, 801, 802, 803, 804, 805, 806, 807, 808, 809, CV1-80, CV1-81, CV1-84, CV1-85, CV1-93, CV1-97, CV1-122, CV1-123, CV1-131 and CV1-135 are novel as such.

The following Tables 1 and 2 summarize all of the experimental data acquired for the molecules G6, A11, G11, F3, H6, F9, H3, F7, H10, NP234 (or 801), NP 246 (or 803), 804, 805, 806, 808, 807, 809, CV1-80, CV1-81, CV1-84, CV1-85, CV1-93, CV1-97, CV1-122, CV1-123, and CV1-135, the structures of which are indicated in columns.

In Table 1, the “code” column corresponds to the name used; the “EC50” column corresponds to the concentration of compound causing a 50% increase (relative to the maximum value) in the percentage of the amplitude of the rapid dissociation rate; the “% al (max)” column corresponds to the maximum value observed for the increase in amplitude of the rapid dissociation rate; the “IC50” column corresponds to the concentration of compound for which a 50% reduction is observed in the binding of 20 nM of NKA-Bo to the EGFP-NK2R receptor; the “Rep Ca” column corresponds to the ability of each compound to evoke a calcium response on cells not expressing the EGFP-NK2R receptor and the “inhib cAMP” column indicates the ability of each compound to inhibit the response of production of cAMP caused by the NKA.

In Table 2, the “code” column corresponds to the name used; the “EC50” column corresponds to the concentration of compound causing a 50% increase (relative to the maximum value) in the percentage of the amplitude of the rapid dissociation rate; the “% al (at 10 μM)” column signifies that the compounds have been tested only at 10 μM and compared to the values determined for 805 (T=22±1 represents the amplitude of rapid dissociation in the absence of effector); the column “% bond reversion (at 10 μM)” signifies that the compounds have been tested only at 10 μM and compared to the values determined for 805.

Amongst all the compounds, 805 has been the subject of a study of the potentialization of the calcium responses caused by the NKA or the truncated NKA on the wild-type human NK2R, wild-type human NK1R, or EGFP-NK2R receptors.

TABLE 1

| | | IUPAC | EC50 | % al | IC50 | Ca | Inhib |
|---|---|---|---|---|---|---|---|
| Code | Structure | name | (μM) | (max) | (μM) | Rep. | cAMP |
| G6 | 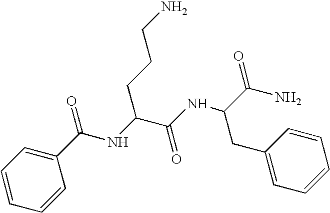 | N-(4-amino-1-(1- carbamoyl-2-phenyl- ethylcarbamoyl)-butyl)- benzamide | ns | ns | 46 | nt | nt |
| A11 | 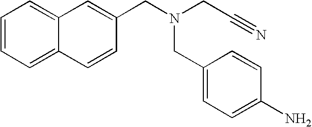 | [(4-Amino-benzyl)- naphthalen-2-ylmethyl-amino]- acetonitrile | 38-44 | ≧90 | 100 | ns | ++ |
| G11 | 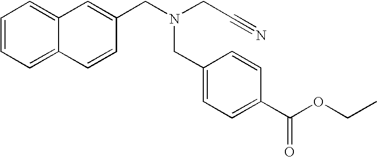 | 4-[(Cyanomethyl-naphthalen-2- ylmethyl-amino)-methyl]- benzoic acid ethyl ester | 10-30 | 70 | >100 | ns | nt |
| F3 | 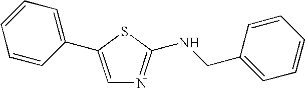 | Benzyl-(5-phenyl-thiazol-2- yl)-amine | 1-5 | 65 | 100 | ++ | + |
| H6 | 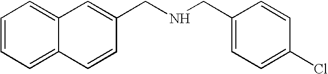 | (4-Chloro-benzyl)- naphthalen-2-ylmethyl- amine | 50-80 | 80 | >100 | ++ | ++ |
| F9 | 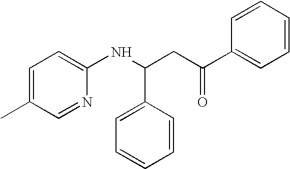 | 3-(5-Methyl-pyridin-2- ylamino)-1,3-diphenyl- propan-1-one | ns | ns | >50 | ++ | nt |
| H3 | 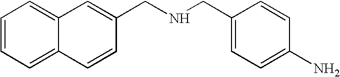 | 4-{[(Naphthalen-2-ylmethyl)- amino]-methyl}-phenylamine | ns | ns | >50 | ns | nt |
| F7 | 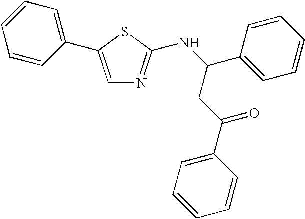 | 1,3-Diphenyl-3-(5-phenyl- thiazol-2-ylamino)-propan-1-one | ≧50 | ≧60 | 90 | ns | nt |
| H10 | 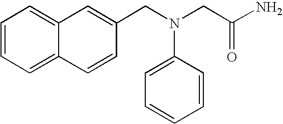 | 2-(Naphthalen-2-ylmethyl- phenyl-amino)-acetamide | >50 | >60 | ≧100 | ns | nt |
| NP234 or 801 | 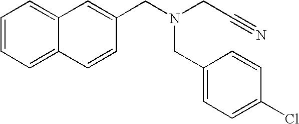 | [(4-chlorobenzyl)(2- naphthylmethyl)amino]acetonitrile | 5-10 | 60-70 | 110 | ns | ++ |
| NP246 or 803 | 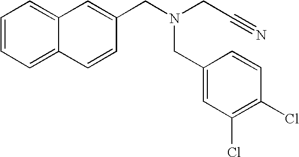 | [(3,4-dichlorobenzyl)(2- naphthylmethyl)amino]acetonitrile | 10-30 | 60-70 | >500 | ns | nt |
| 804 | 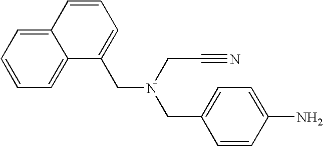 | [(4-aminobenzyl)(1- naphthylmethyl)amino]acetonitrile | 20-30 | ≧80 | >100 | ns | nt |
| 805 | 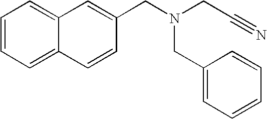 | [benzyl(2-naphthylmethyl) amino]acetonitrile | 3-8 | 80 | >500 | ns | ++ |
| 806 | 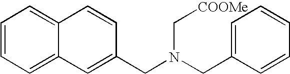 | methyl [benzyl(2- naphtylmethyl)amino]acetate | 10-20 | 90 | >200 | ns | nt |
| 808 | 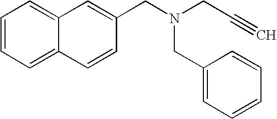 | N-benzyl-N-(2- naphthylmethyl)-N-prop-2- ynylamine | 20-30 | ≧70 | >200 | + | nt |
| 807 | 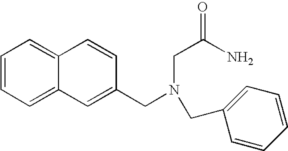 | 2-[benzyl(2- naphthylmethyl)amino]acetamide | ? | ≧90 | 100 | +? | nt |
| 809 | 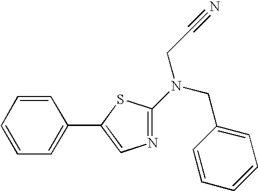 | [benzyl(5-phenyl-1,3- thiazol-2-yl)amino]acetonitrile | 1-20 | ≧75 | >100 | nt | nt |

ns: not significant; nt: not tested: ?: uniterpretable

TABLE 2

| | | | | % al | % bond | |
|---|---|---|---|---|---|---|
| | | IUPAC | EC50 | (at 10(μM) | reversion | Ca |
| Code | Structure | name | (μM) | (T = 22 ± 1 | (at 10 μM) | Rep. |
| 805 | | | 8 | 29 (10 μM) | 1 (10 μM) | ns |
| | | | | 45 (50 μM) | 9 (50 μM) | |
| CV1-80 | 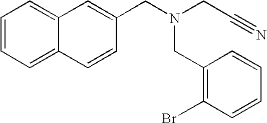 | [(2-bromobenzyl)(2- naphthylmethyl)amino]acetonitrile | nt | 25 | 16 | nt |
| CV1-81 | 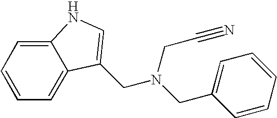 | [benzyl(1H-indol-3- ylmethyl)[amino]acetonitrile | nt | 22 | 15 | nt |
| CV1-84 | 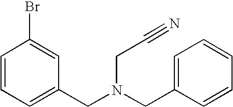 | [benzyl(3- bromobenzyl)amino]acetonitrile | nt | 25 | 4 | nt |
| CV1-85 | 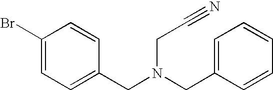 | [benzyl(4- bromobenzyl)amino]acetonitrile | nt | 28 | 4 | nt |
| CV1-93 | 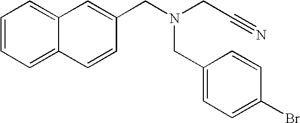 | [4-bromobenzyl(2- naphthylmethyl)amino]acetonitrile | nt | 26 | 3 | nt |
| CV1-97 | 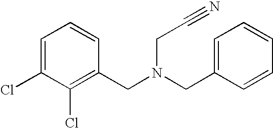 | [benzyl(2,3- dichlorobenzyl[amino]acetonitrile | nt | 34 | 15 | nt |
| CV1-122 | 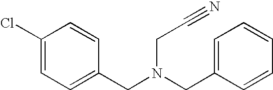 | [benzyl(4- chlorobenzyl[amino]acetonitrile | nt | 31 | 17 | nt |
| CV1-123 | 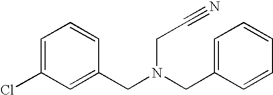 | [benzyl(3- chlorobenzyl[amino]acetonitrile | nt | 26 | 7 | nt |
| CV1-135 | 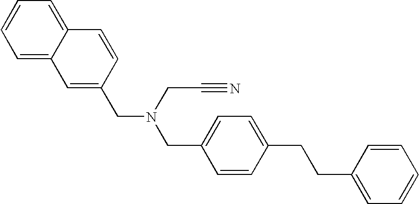 | | nt | 22 | 6 | nt |

ns: not significant; nt: not tested; ?: uninterpretable

The abovementioned compounds are divided into two large families: Family I and Family II.

Family I includes the compounds A11, G11, H10, 801, 803, 804, 805, 806, 808, 807, CV1-80, CV1-81, CV1-84, CV1-85, CV1-93, CV1-97, CV1-122, CV1-123 and CV1-135.

These compounds correspond to the following general formula:

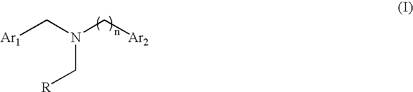

in which n is equal to 0 or 1, Ar₁ and Ar₂ represent monocyclic or bicyclic substituted aromatic groups and R represents an electroattracting group such as CN, COOMe or CONR₁R₂ or an unsaturated group such as an alkene or alkyne group.

| compound | n | Ar₁ | Ar₂ | R |
|---|---|---|---|---|
| A11 | 1 | β-naphtyl | 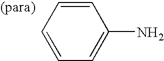 | 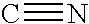 |
| G11 | 1 | β-naphtyl | 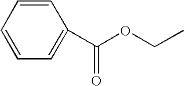 | 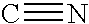 |
| H10 | 0 | β-naphtyl | 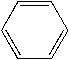 | CONH₂ |
| 801 | 1 | β-naphtyl | 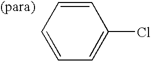 | 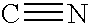 |
| 803 | 1 | β-naphtyl | 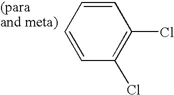 | 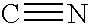 |
| 804 | 1 | α-naphtyl | 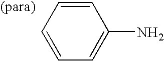 | 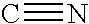 |
| 805 | 1 | β-naphtyl | 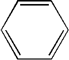 | 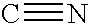 |
| 806 | 1 | β-naphtyl | 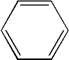 | COOCH₃ |
| 808 | 1 | β-naphtyl | 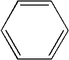 | 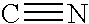 |

The compounds H6 and H3 are synthesis intermediates making it possible to obtain compounds of the abovementioned Family I.

Family II includes the compounds F3, F7 and 809.

These compounds correspond to the following general formula:

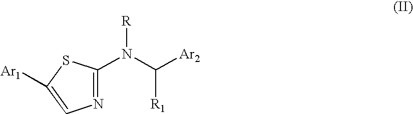

in which Ar₁ and Ar₂ represent aromatic groups, R represents either a hydrogen atom or a —CH₂CN group, and R₁ is a hydrogen atom or a CH₂COφ group.

The compounds F7 and F9 are already known and are described in the article by Moutou et al. (1994).

Moreover, the compound G6 of Table 1 is already known and described in the International Application WO 02/24192.

Methods for the Preparation of the Abovementioned Compounds:

I—Method for the Preparation of the Compounds of Family I:

A) First Stage: Preparation of the Secondary Amines:

The primary amine (1 eq) (see table hereafter) is added to a solution of the appropriate aromatic aldehyde (1 eq) in methanol. The reaction medium is heated at 60° C. for two hours then cooled down rapidly to 0° C. Then NaBH₄ (2 eq) is added and the reaction medium is stirred for a few minutes then returned to ambient temperature over one hour. After evaporation of the solution, extraction is carried out with AcOEt by washing with water then with a saturated solution of NaCl, followed by drying with Na₂SO₄, filtering on frit and evaporation to dryness. The secondary amine, thus isolated, most generally in the form of an oil, is used as it is without any purification.

The secondary amine thus obtained corresponds to the following formula:

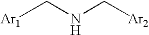

in which Ar₁ and Ar₂ are as defined above.

An example of a reaction corresponding to Stage 1 (here obtaining compound H3) is:

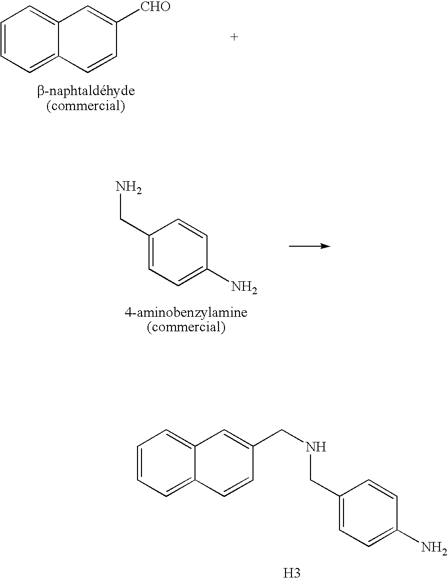

Table summarizing the starting products:

| Final | | |
|---|---|---|
| product | | |
| obtained at | | |
| the end of | Starting | Starting |
| Stage 2 | aldehyde | amine |
| H3 | β-naphthaldehyde | 4-aminobenzylamine |
| A11 | β-naphthaldehyde | 4-aminobenzylamine |
| G11 | β-naphthaldehyde | 4-ethoxycarbonylbenzylamine |
| H6 | β-naphthaldehyde | 4-chlrobenzylamine |
| H4 | β-naphthaldehyde | benzylamine |
| H10 | β-naphtha1dehyde | aniline |
| 801 | β-naphthaldehyde | 4-chlorobenzylamine |
| 803 | β-naphthaldehyde | 3,4-dichlorobenzylamine |
| 804 | α-naphthaldehyde | 4-aminobenzylamine |
| 805 | β-naphthaldehyde | benzylamine |
| 806 | β-naphthaldehyde | benzylamine |
| 808 | β-naphthaldehyde | benzylamine |
| 807 | β-naphthaldehyde | benzylamine |
| CV1-80 | β-naphthaldehyde | 2-bromobenzylamine |
| CV1-81 | β-naphthaldehyde | indol 3-methylamine |
| CV1-84 | 3-bromobenzaldehyde | benzylamine |
| CV1-85 | 4-bromobenzaldehyde | benzylamine |
| CV1-93 | β-naphthaldehyde | 4-bromobenzylamine |
| CV1-97 | 3,4-dichlorobenzaldehyde | benzylamine |
| CV1-122 | 4-chlrobenzaldehyde | benzylamine |
| CV1-123 | 3-chlrobenzaldehyde | benzylamine |

B) Second Stage: Preparation of the Tertiary Amines:

The secondary amine (1.1 eq) obtained in the preceding stage is dissolved in dimethylformamide, and alkyl halide (chloroacetonitrile, propargyl chloride, etc.) (see below) is added. The solution is heated to reflux for 12 hours then left to return to ambient temperature, followed by extraction with ether (10 times more in volume than the DMF) and washing with water then with a saturated solution of NaCl. The organic phase is dried with Na₂SO₄ then it is evaporated to dryness. The crude product is purified by silica gel chromatography (AcOEt 1/Hex 9). The hydrochlorides are prepared by bubbling gaseous HCl through the AcOEt.

The abovementioned compounds are then obtained, in the form of the hydrochloride, of formula as defined below:

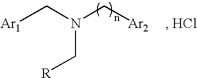

More precisely, this second stage corresponds to the following reaction diagram:

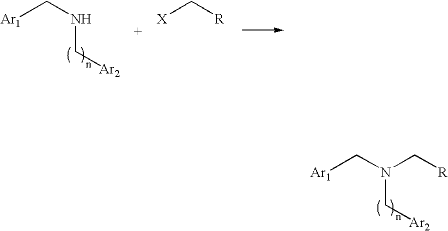

The secondary amines are therefore reacted with different commercial halides.

For the compounds A11, G11, 801, 803, 804, 805, CV1-80, CV1-81, CV1-84, CV1-85, CV1-93, CV1-122, CV1-123 and CV1-135, chloroacetonitrile is used.

For the compounds 806 and CV1-97, bromo or chloroethyl or methyl acetate is used.

For the compounds H4, H10 and 807, bromo or chloroacetamide is used.

For compound 808, propargyl bromide or chloride is used.

Concerning compound CV1-135, it is obtained according to a particular operating method, as described hereafter:

4-bromobenzonitrile of formula: 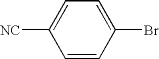
as well as the catalyst PdCl₂(PPh₃)₂, copper iodide CuI, the base NEt₃ and phenylacetylene are dissolved in acetonitrile. The reaction medium is heated overnight at 50-60° C., followed by evaporation at the end of reaction and purification by chromatography on a silica column.

This operating method corresponds to the following reaction diagram:

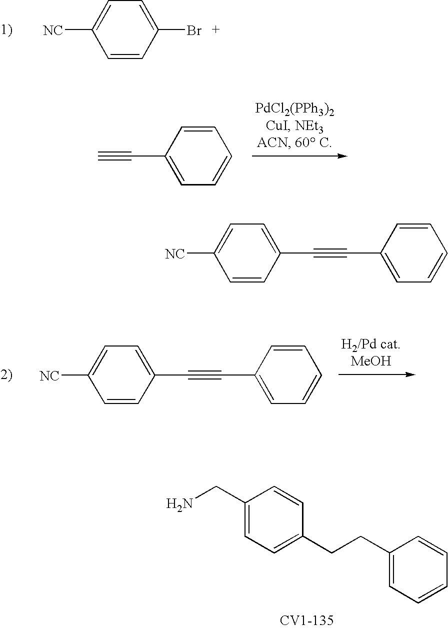

II—Method for the Preparation of the Compounds of Family II:

5-phenyl 2-benzylaminothiazole is obtained starting with 5-phenyl-2-aminothiazole by condensation with benzaldehyde, followed by hydrogenation. The compound obtained is then alkylated by chloroacetonitrile (see operating method I).

Compound F3 is obtained according to the following reaction diagram:

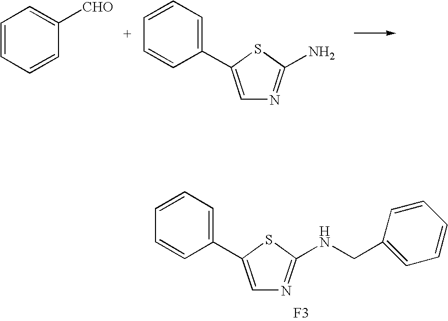

NaH (1.3 eq) is added to a solution of 5-phenyl-2-aminobenzylthiazole (1 eq) in DMF cooled down to 0° C. The reaction medium is stirred for 15 minutes then chloroacetonitrile (1 eq) is added dropwise. Stirring is continued at ambient temperature for 8 hours, the solution is then diluted with water. After extraction with ethyl acetate, the organic phase is dried over anhydrous sodium sulphate and evaporated. The crude reaction product is purified by silica gel chromatography (AcOEt 1/Hexane 2).

Thus for example compound 809 is therefore obtained: ((5-phenyl-thiazol-2-yl)-N-benzyl)acetonitrile C₁₈H₁₅N₃S (305.40 g·mol⁻¹) in the form of a beige powder, according to the following reaction diagram:

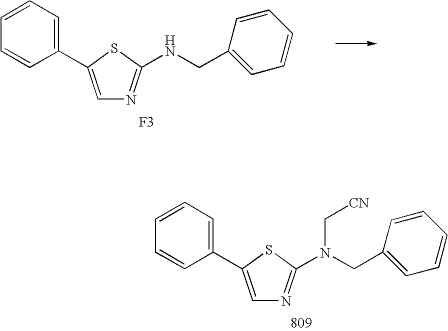

NMR ¹H (CDCl₃, 200 MHz): 4.42 (s, 2H); 4.70 (s, 2H); 7.30-7.51 (m, 11H)

### DETAILED DESCRIPTION OF THE INVENTION

In its preferred embodiment, the development of the invention uses the cDNA coding for the green fluorescent protein (Prasher et al., 1992) of the jellyfish Aequorea victoria, preferentially the mutants EYFP, EGFP and ECPF of this protein optimized for their expression in the preferred host organisms, mammal cells.

The cDNA can be modified in order to code for a variant in which one or more amino acids are substituted, inserted or deleted in order to allow its N- or C-terminal fusion with the gene coding for a receptor.

The receptor can be chosen from:

1) the receptors of neurotransmitters coupled to G proteins structurally linked to the adrenergic receptors and metabotropic receptors of glutamate as presented in the list which is updated annually and published in the GPCRdb (http://www.gcrdb.uthscsa.edu or http://www.gpcr.org), and Ensembl databases (http://www.ensembl.org) inter alia.

2) the receptor-channels structurally linked to the nicotinic receptors, to the glutamate receptors and to the ATP receptors, as presented in the list which is updated annually and published in the GPCRdb (http://www.gcrdb.uthscsa.edu or http://www.gpcr.org), and Ensembl databases (http://www.ensembl.org) inter alia.

3) the nuclear receptors possessing a DNA interaction domain structurally linked to the steroids receptor (Mangelsdorf et al., 1995; Wurtz et al., 1996).

4) the receptors of the plasma membrane with tyrosine kinase activity structurally linked to the insulin receptor (Yarden, Y. and Ullrich, A., 1988).

5) the membrane receptors coupled to the protein tyrosine kinases (STATs, TYK2, Jak) structurally linked to the γ interferon receptor (Brisco et al., 1996; Ihle, 1995).

In the case where the fusion is carried out between the EGFP and a receptor coupled to the G proteins (group 1), the fusion can be carried out in particular:

1) on the N-terminal side of the receptor, and therefore on the C-terminal side of the EGFP,

2) on the C-terminal side of the receptor and therefore on the N-terminal side of the EGFP,

3) in the sequence of the receptor, in particular in the first or third intracellular loop, optionally by introducing one or more copies of a spacer sequence, in particular -GGGGS-.

In the case where the fusion is carried out between the EGFP and a receptor-channel (group 2), the fusion can be carried out in particular:

1) in the region homologous to the “major immunogenic region” of the a sub-unit of the Torpedo nicotinic receptor (residues 67-76), optionally by introducing one or more copies of a spacer sequence, in particular -GGGGS-.

In the case where the fusion is carried out between the EGFP and a nuclear receptor (group 3), the fusion can be carried out in particular:

1) on the N-terminal side of the receptor, and therefore on the C-terminal side of the EGFP,

2) on the N-terminal side of the receptor, truncated in its N-terminal part upstream of the DNA-binding domain, and therefore on the C-terminal side of the EGFP.

In the case where the fusion is carried out between the EGFP and a receptor either with tyrosine kinase activity, or coupled to a tyrosine kinase (groups 4 and 5), the fusion can be carried out in particular:

1) on the N-terminal side of the receptor, and therefore on the C-terminal side of the EGFP.

Any gene coding for a fluorescent protein, in particular GFP, coupled to a receptor, and deriving from organisms expressing GFP or similar proteins could be used in this invention.

The DNA sequences coding for GFP and the target proteins, in particular receptors, can be of genomic origin or can be cDNAs, and can be obtained from the DNA of any eukaryotic or prokaryotic, animal or plant species, for example by preparing gene banks or cDNA banks and by screening these banks in order to identify the coding sequences by hybridization with oligonucleotide probes by standard techniques (Current Protocols in Molecular Biology, op. cit.).

The DNA constructions coding for GFP and the target proteins can also be obtained by total synthesis by the standard methods, in particular the phosphoramidite method (Beaucage and Caruthers, 1981) and the use of automated DNA synthesis apparatus, the polynucleotides obtained then being purified, ligated and cloned in the appropriate vectors. For most applications, the genes coding for GFP and the target proteins are preferentially obtained by screening banks, whereas the spacer arms as well as the oligonucleotides required for the mutagenesis are preferentially obtained by synthesis.

The DNA constructions can be of mixed, synthetic and genomic nature, by ligation of synthetic fragments with elements from genomic DNA, according to standard procedures (Current Protocols in Molecular Biology, op.cit.).

The DNA constructions can also be obtained by PCR (“polymerase chain reaction”) by using specific primers, such as for example described in PCR protocol 1990, Academic press, San Diego, Calif., USA.

Finally, the DNA constructions can be modified by other methods including for example chemical reactions, random or directed mutagenesis, by insertion, deletion or substitution of nucleotides, these modifications being able to alter properties of one protein or another, in particular, GFP and the target proteins.

The DNA constructions can be inserted into a recombinant vector. This vector can be any appropriate vector for the procedures used with recombinant vectors. The choice of the vector is often carried out as a function of the host cell in which the DNA construction is to be introduced. The vector can thus be a vector capable of replicating in autonomous, i.e. extrachromosomal, manner and independent of the chromosomal replication, for example a plasmid. Alternatively, the vector can be developed in order to integrate all or part of the DNA that it contains into the genome of the host cell, and will be replicated at the same time as the chromosome(s) into which it is integrated.

The vector is preferentially an expression vector in which the GFP fused with the receptor or the GFP fused with the ligand is under the control of other DNA segments required for the transcription. In general, the expression vector derived from plasmidic or viral DNA can contain elements of one and the other.

The term “under the control” indicates that the DNA segments are arranged on the vector so that they function in concert in order to serve the desired objective, for example, the transcription is initiated in the promoter and continues throughout the sequence coding for the receptor fused to the GFP or the ligand fused to the GFP.

The promoter can be any DNA sequence capable of promoting a transcriptional activity in the host cell chosen and can be derived from genes homologous or heterologous to the host cell.

Examples of promoters suitable for the expression of the receptor fused with the GFP or of the ligand fused with the GFP in mammal cells are the simian virus SV40 promoter (Subramani et al., 1981), the Rous sarcoma virus (RSV) promoter, the cytomegalovirus (CMV) promoter or the adenovirus major late promoter (AdMLP).

Examples of Promoters for Insect Cells:

Polyhedrin promoter (U.S. Pat. No. 4,745,051; Vasuvedan et al., 1992) P10 promoter (Vlack et al., 1988), the promoter of the early 1 gene of the baculovirus (U.S. Pat. No. 5,155,037; U.S. Pat. No. 5,162,222).

Examples of Promoters for Yeasts:

Promoters of the genes of glycolysis (Hitzeman et al., 1980; Alber and Kawasaki, 1982), of the alcohol dehydrogenase genes (Young et al., 1982).

Examples of Promoters for Bacteria:

Examples of promoters for expression in bacteria can be constitutive promoters such as the polymerase T7 promoter, or inducible promoters such as for example the phage lambda pL promoter (Current Protocols in Molecular Biology, op.cit.).

Examples of Promoters for Filamentous Fungi:

The promoters which can be used are for example the ADH3 promoter (McKnight et al., 1985) or the tpiA promoter. Other useful promoters can be derived from the genes coding for the aspartate proteinase of Rhizomucor miehei, the neutral alpha-amylase of Aspergillus niger, the acetamidase of Aspergillus nidulans, the TAKA amylase of Aspergillus oryzae or the glucoamylase promoter of Aspergillus awamori.

The vector can moreover contain:

polyadenylation sequences, such as for example those of SV40 or of the Elb 5 region of the adenovirus,

transcription activating (enhancer) sequences (SV40 activator),

replication sequences such as for example the replication sequences of SV40 or of the Epstein Barr virus, for mammal cells or the origin and the replication genes REP 1-3 of the plasmid 2μ, for yeasts,

selection markers, namely genes conferring resistance to an antibiotic (neomycin, zeocin, hygromycin, ampicillin, kanamycin, tetracyclin, chloramphenicol etc.) or allowing compensation for a fault (gene coding for dihydrofolate reductase allowing resistance to methotrexate, or TPI gene of S. pombe described by Russell (1985).

The host cell can be any cell capable of expressing the DNA construction inserted into an appropriate vector.

The cells can be in particular bacteria, yeasts, fungi and higher eukaryotic cells such as for example mammal cells.

Examples of bacterial cells capable of expressing the DNA constructions are:

gram-positive bacteria such as Bacillus strains such as B. subtilis, B. licheniformis, B. lentus, B. brevis, B. strearothermophilus, B. thurigiensis or Streptomyces strains such as S. lividans, S murinus,

gram-negative bacteria such as Escherichia coli.

The transformation of the bacteria can be carried out by protoplastic transformation or by transformation of competent bacteria (Current Protocols in Molecular Biology, op.cit.).

Examples of Eukaryotic Cells:

HEK 293, HeLa cell lines, primary cultures, COS cells (for example ATCC CRL 1650), BHK cells (for example ATCC CRL 1632) CHO cells (for example ATCC CCL 61).

The methods for introducing DNA into these cells (transfection, lipofection, electroporation etc.) are described in Current Protocols in Molecular Biology, op.cit.

Examples of Yeast Cells:

Saccharomyces, S. cerevisiae, S. kluyveri,

Kluiveromyces, K. lactis,

Hansenula, H. polymorpha,

Pichia, P. pastoris,

transformed by introduction of heterologous DNA according to the protocols described in Current Protocols in Molecular Biology, op.cit.

The transformed cells are selected by a phenotype determined by a resistance marker, generally to a drug, or by their ability to proliferate in the absence of a particular nutrient.

Examples of Filamentous Fungi:

The Aspergillus strains (A. oryzae, A. nidulans, A. niger), Neurospora, Fusarium, Trichoderma. The use of Aspergillus for the expression of proteins is described in EP 272 277 or EP 230 023 or EP 184 438.

Examples of Insect Cells:

There can be mentioned the lines of Lepidoptera e.g. Spodoptera frugiperda (Sf9) or Trichoplusia ni (Tni). The transformation methods (infection in particular) are described in Current Protocols in Molecular Biology (op.cit.).

The Ligands

The ligands interacting with the receptor can be of any origin (natural, synthetic, semi-synthetic, recombinant), and any structure (chemical, peptidic, proteic). They can be naturally fluorescent (or carriers of a chromophore) or can require either a chemical reaction allowing the grafting of a fluorescent group (or fluorescent group precursor) or of a chromophore, or a DNA construction leading to the fusion of the ligand with the GFP and allowing the expression of the ligand thus rendered fluorescent.

Examples of Chemical Reactions are:

coupling of amines or thiols with reagents of alkyl halide, aryl halides, acyl halides, acid halide type, the isothiocyanate group, the maleimide group, the epoxides, in an organic solvent in the presence of a base or in aqueous medium,

coupling of acids with amines activated by groups such as the succinimides.

According to the process of the invention, the fluorescence of the transformed cells can be measured in a spectrofluorimeter with which the spectral properties of the cells, in suspension or adherent, can be determined by the acquisition of their excitation and emission spectra. The interactions with the fluorescent ligand are then detected by changes in the excitation and/or emission spectra of the energy donor and acceptor, and the ligands are defined as pharmacologically significant if their interactions with the receptor are inhibited by the addition of an excess of non-fluorescent ligand preventing the interaction between the fluorescent receptor and the fluorescent ligand.

Binding Measurements

Measurements of the association and/or dissociation kinetics are carried out by all the means making it possible to record the formation or dissociation of the complex between the marked ligand and the marked receptor, in continuous or discontinuous manner, such that the association and dissociation kinetic parameters are determined, namely the rate constant(s), as well as the relative amplitude(s) associated with each association and/or dissociation kinetics stage.

In its preferred embodiment, the kinetics, the apparent rate constants of which are above 0.1 s⁻¹, are recorded using a rapid mixing device connected to an excitation and fluorescence detection device (FIG. 1). The samples to be mixed are arranged in mixing syringes (or other containers), then mixed rapidly. In this context, “rapidly” means that the content of the syringes is driven at a rate greater than or equal to 4 ml/sec for a 100 μl observation chamber, as described for example in Palanché et al. (2001).

In the case where the apparent rate constants are below 0.1 s⁻¹, the use of a rapid mixing device is not essential. In this case it is possible to record the variations in the fluorescence of the donor and/or acceptor in a standard spectrofluorimeter provided with a magnetic stirring sample device, temperature regulation and a recording function over time. The samples are then arranged in a cuvette equipped with a stirrer and the mixture is obtained by the manual addition of the desired components or solutions. The samples containing the receptor can be either cells, or fragments of cells. They are preferentially arranged first in the cuvette and the modulating agents as well as the marked ligand are added to this solution. The effect of these additions can thus be recorded and any variation in fluorescence can be characterized at the physical and pharmacological level in order to determiner whether or not they are involved in the energy transfer process and whether or not they correspond to the criteria of the sought pharmacological specificity. In its preferred embodiment, a dissociation measurement is obtained by adding an excess of competitive ligand. This can be obtained:

by adding a small volume (≦5% of the final volume) of a concentrated solution of competitive ligand (approximately 500-1000 times its K_{d} in the final solution, the K_{d} corresponding to the concentration leading to 50% occupation of the sites) in a cuvette containing a mixture of fluorescent ligand, fluorescent receptor and optionally allosteric effector, or

by rapid mixing of the content of a syringe containing the fluorescent ligand, the fluorescent receptor and optionally the allosteric effector with that of a syringe containing the competitive ligand at the desired concentration as well as the allosteric effector at a concentration equal to that of the other syringe (for a volume to volume mixture) in order to avoid diluting it during the mixing operation.

In the preferred embodiment of the process, the association measurements are carried out by mixing the fluorescent ligand with cells, membranes or extracts, containing the fluorescent receptor with the fluorescent ligand, while monitoring the variation in fluorescence due to the energy transfer. The effect of the effector can be studied after previous mixing with the receptor or in a protocol of simultaneous mixing with the fluorescent ligand. The mixing can be carried out by means of a rapid mixing device or by means of manual mixing in a spectrofluorimeter cuvette.

Response Measurements

Given the diversity of receptors and the variable number of the conformational states that they can adopt, it is the measurement of the modulation of the responses which makes it possible to define the positive or negative character of the allosteric effector.

It is recalled that in this context, the positive or negative character of an allosteric effector is defined by its ability to potentialize (positive effector) or to depress (negative effector) a physiological response specific to the receptor studied. The positive allosteric effector is considered as such, whatever its effect (acceleration or deceleration) on the association and/or dissociation kinetics of the fluorescent marker ligand, whether the latter is agonist or antagonist.

The different tests used to measure the responses have been given above.

All the parameters of a response can be affected by an positive allosteric effector. These parameters include inter alia, the rate of establishment of the response, its amplitude, its duration, its frequency, its sensitivity to the agonist and the base level of the response.

In its preferred embodiment, the process is intended for the study of the receptors coupled to the G proteins. For these receptors, the responses being able to be the subject of a study are varied as they are coupled to multiple signal transduction routes. There can be mentioned for example the binding of GTP to the G protein, the production of cAMP, inositol phosphates, arachidonic acid, the phosphorylation of proteins, the release of intracellular calcium, the modification of cell pH, the modification of the cell proliferation rate, the alteration of the cell morphology, the polymerization of actin, or also the regulation of ionic channels or that of gene expression.

Each of these responses can be recorded and the effects of the effector can be determined.

In the most widespread cases, the primary focus is on the calcium responses and the production of cAMP.

For the calcium responses, it is possible to use an optical probe sensitive to calcium or electrophysiological recording of the regulation of currents. The use of a calcium probe makes it possible to determine the delay in establishment of the response, the duration of the response, its intensity or its sensitivity to the agonist. The electrophysiological recording moreover allows analysis of the frequency of the opening of channels (Mulle et al., 1992). A positive allosteric effector can reduce the delay in establishment of the response and increase the other parameters (amplitude, duration, sensitivity etc.). The negative allosteric effector will have contrary effects.

### DESCRIPTION OF THE FIGURES

FIG. 1 shows the diagram of a rapid mixing device making it possible to carry out the rapid kinetics measurements. The device is constituted by two syringes, a mixing chamber and an observation chamber. The advance of the syringe pistons allows the mixture of the content of the syringes in the mixing chamber. After a dead time, the mixture arrives in the observation chamber equipped with an excitation and fluorescence detection device. Stopping the thrust of the pistons, or the arrival of the stopping syringe at the stop, halts the mixing stage, on completion of which the evolution of the mixture is recorded.

FIG. 2 (taken from Monod, Wyman and Changeux, 1965) represents a diagram of regulating protein existing in two conformational states or two oligomerization states. In this model, the protein can exist in several discrete states, in a finite number, which correspond to thermodynamically stable states which differ from each other by their tertiary and quaternary structure. The interconversion between each state can operate spontaneously and is described by the isomerization parameter.

The conformations differ in their pharmacological properties. Thus, the ligands stabilize the conformations for which they exhibit the highest affinity. The conformations differ in their functional properties. Thus the agonists preferentially stabilize the active state, whereas the antagonists preferentially stabilize the inactive state.

In this diagram, the relaxed state correspond to the “active” conformational state of the protein. This conformation can bind, with a high affinity, the agonists (A) if it is a receptor or the substrates (S) if it is an enzyme. The constrained and/or monomeric states are less (or not) active in comparison to the relaxed state, which is the most active. The constrained state binds with a high affinity the inhibitors or antagonists. The monomeric state binds with a moderate affinity the agonists or the substrates.

FIGS. 3A and 3B show a quantified diagram of the ratios between the conformational states and the interactions with the ligands.

In FIG. 3A, the protein exists in two conformations corresponding to, a quiescent state (non-active at biological level) and an active state. The spontaneous isomerization between the R and A states is described by the isomerization constant L₀ equal to the ratio of the relative concentrations of each state (L₀=[R]/[A]). The ligands of the protein bind to the R state with a dissociation constant equal to K_{R} and to the A state with a dissociation constant equal to K_{A}. The ratio c=K_{A}/K_{R} describes the affinity ratio for a given ligand between the conformations. If c is less than 1, the ligand is agonist (it increases the fractional concentration of A). If c is less than or equal to 1, the ligand is antagonist (it increases the fractional concentration of R). The equilibrium between the R and A conformations bound to the ligand (RF [symbol] AF) is described by the product of L₀ by c.

In the presence of an allosteric effector (see FIG. 3B), the isomerization constant L₀ is altered to L₀′ according to L₀′=L[(1+βd)/(1+β)]ⁿ where β (homologue of c) describes the affinity ratio of the effector for the two conformations (β=K_{A}/K_{R}) and d is the concentration of effector standardized relative to the affinity for the A state (d=[effector]/K_{A}). The effectors of which β>1 are inhibitors, those of which β<1 are potentializers.

FIG. 4 represents a recording in real time of the association (A) and dissociation (B) kinetics of NKA-Bo (NKA marked by Bodipy) with the receptor EGFP-NK2R (NK2R marked by GFP). The recording of the association kinetics is carried out after rapid mixing of HEK 293 cells expressing the receptor EGFP-NK2R. The dissociation kinetics are recorded after manual mixing of the cells expressing the receptor with the NKA-bo (pre-incubation for 15 minutes, 100 nanomolars) then with the competitive antagonist SR 4896 (10 μM final).

The x-axis represents time in seconds and the y-axis represents fluorescence (arbitrary units) at 510 nm. The values λn and κn correspond respectively to the amplitudes and to the rate constants determined by a least adjustment squares with two exponentials.

FIGS. 5A, 5B, 5C and 5D indicate the procedure for identification of a ligand interacting in a competitive manner with the fluorescent ligand on a receptor site.

The complex formed between the receptor EGFP-NK2R and the NKA-Bo is reversed by increasing concentrations of the molecule G6 (see Table 1) then by the non-fluorescent NKA.

FIG. 5A indicates that part of the receptor-ligand complex is reversed during each addition of molecule G6 (10 or 20 μM), as well as during the addition of a saturating concentration of NKA (10 μM). In this figure, the x-axis corresponds to time in seconds and the y-axis to the fluorescence measured at 510 nm.

FIG. 5B shows that the dissociation kinetics of the NKA-Bo, determined after addition of a saturating concentration of NKA are not modified by the presence of the molecule G6.

The two experimental traces are adjusted using 2 rapid (0.04 s⁻¹) and slow (0.014 s⁻¹) exponentials the amplitudes (66% for the rapid and 33% for the slow) of which are not affected by the presence of 50 μmoles of molecule G6.

In this figure, the x-axis corresponds to time in seconds and the y-axis to the fluorescence measured at 510 nm.

FIG. 5C represents a quantitative study of the dissociation kinetics of NKA-Bo in the presence of a known competitive antagonist, SR 48968. The dissociation of NKA-Bo is recorded in the presence of 0.2; 1 and 5 nM of SR 48968. Under the three experimental conditions, the dissociation rate can be described as a sum of two exponentials the rates of which are 0.04 sec⁻¹ and 0.008 sec⁻¹, and the relative amplitudes of which remain constant at the different doses of SR 48968 (45% of rapid dissociation (0.04 s⁻¹) and 55% of slow dissociation (0.008 s⁻¹)).

In this figure, the x-axis corresponds to time in seconds and the y-axis to the fluorescence measured at 510 nm.

FIG. 5D represents the values of the amplitudes of rapid (squares) and slow (upward-pointing triangles) dissociation of NKA-Bo in the presence of SR 48968, as well as the total amplitude of the binding of NKA-Bo (downward-pointing triangles).

In this figure, the x-axis corresponds to the logarithm of the SR 48968 concentration and the y-axis to the amplitude of the standardized fluorescence.

The upper part of FIG. 5D represents the ratio of the amplitude of the slow dissociation (A2) relative to the total amplitude of dissociation (total A) (curve with the diamonds). It is noted that this ratio is constant. In this graph, the x-axis corresponds to the logarithm of the SR 48968 concentration and the y-axis to the percentage of the amplitude of the fluorescence.

FIGS. 6A, 6B and 6C indicate the procedure for identification of a ligand interacting in a non-competitive manner with the fluorescent ligand on a receptor site.

FIG. 6A shows the fluorescence measured at 510 nm as a function of time in seconds.

The complex formed between the receptor EGFP-NK2R and the NKA-Bo is reversed by increasing concentrations of the molecule A11 then by the non-fluorescent NKA. FIG. 6A indicates that a small portion of the receptor-ligand complex is reversed during each addition of molecule A11, whereas the NKA (10 μM) reverses the majority of the complex. The kinetics of the dissociation by NKA is not in the same form as in the control.

FIG. 6B shows the fluorescence measured at 510 nm as a function of time in seconds. FIG. 6B illustrates the modification of dissociation kinetics in the presence of molecule A11. The recordings of dissociation of NKA-Bo from its receptor EGFP-NK2R are carried out in the presence and in the absence of A11. The dissociation is initiated by the addition of non-fluorescent NKA at a final concentration of 20 μM. Analysis of the dissociation kinetics shows two monoexponential relaxations the rate constants (k1 and k2) of which are identical in the presence and in the absence of A11 but the relative amplitudes of which change such that the slow dissociation represents 59% of the total signal in the control and 22% of the total signal in the presence of A11.

FIG. 6C shows the association of NKABo (20 nanomolars) with the receptor EGFP-NK2R, as a function of time in seconds. FIG. 6C represents recordings of time-resolved binding (association) of NKA-Bo to its receptor EGFP-NK2R, in the absence and in the presence of the molecule A11. Analysis of the binding kinetics shows two monoexponential relaxations characterized by rate constants (k1 and k2) not changing in the presence of A11, but of variable amplitudes according to whether or not the cells are incubated with A11.

FIGS. 7A to 7I illustrate the analysis of the effects of various molecules on the dissociation of NKA-Bo bound to its fluorescent receptor EGFP-NK2R.

FIG. 7A represents the recordings of dissociation of NKA-Bo in the presence of variable concentrations (1, 10 and 50 μM) of the 805 molecule (see Table 1). The fluorescence measured at 510 nm is represented as a function of time in seconds. Quantitative analysis of the dissociation kinetics is obtained using two rapid (0.04 sec⁻¹) and slow (0.008 sec⁻¹) exponentials the relative amplitudes (rapid/slow) of which vary from 30/70 to 80/20 when the concentration of 805 increases.

FIGS. 7B to 71 show the percentage of the amplitude of rapid dissociation as a function of the logarithm of the concentration of the compounds tested. These figures give the results of the quantitative analysis of the dissociation of NKA-Bo in the presence of the 805 (FIG. 7B), NP246 (FIG. 7C), A11 (FIG. 7D), H10 (FIG. 7E), G11 (FIG. 7F), F7 (FIG. 7G), F3 (FIG. 7H) and NP 234 (FIG. 71) molecules. The dissociation of NKA-Bo was determined in the presence of each molecule at the concentrations indicated on the x-axis. The effect of an increase in the dissociation rate is represented by the relative increase in the amplitude (% λ 1) of rapid dissociation.

FIGS. 8A and 8B show a quantification of the shift of [³H]SR 48968, bound to the receptor EGFP-NK2R, by the NKA and the 805 molecule.

These figures show the binding of 1 nanomolar [³H]SR 48968 (Amersham, Life Sciences) as a function of the logarithm of the NKA concentration (FIG. 8A) or as a function of the 805 molecule concentration (FIG. 8B).

It is noted that the NKA (competitive ligand) reverses 100% of the binding of [³H]SR 48968 whereas 805 (non-competitive ligand) only reverses approximately 35% of the binding of SR 48968.

FIGS. 9A, 9B and 9C show the results of stimulation of production of cAMP by the NKA in HEK 293 cells expressing the receptor EGFP-NK2R.

FIG. 9A represents the effect of the presence of competitive antagonist (H8565) and allosteric effector (805). This figure shows the accumulation of intracellular cAMP (in pmoles/well) as a function of the logarithm of the NKA concentration. The curve with the black squares corresponds to the blank; the curve with the black triangles corresponds to the antagonist H8565 at a concentration of 1 μM; the curve with the grey squares corresponds to the ligand 805 at a concentration of 10 μM and the curve with the grey triangles corresponds to the ligand 805 at a concentration of 50 μM.

The accumulation of intracellular cAMP is determined at different NKA concentrations. The antagonist H8565 shifts the NKA concentration-response curve towards higher values without affecting the intensity of the maximum response (black triangles). The ligand 805 (grey squares and triangles) reduces the intensity of the maximum response without affecting the EC₅₀ (50%-effective concentration) of the NKA for the response.

FIG. 9B represents the 50%-effective concentration of the maximum response of cyclic AMP to the NKA. This EC₅₀ increases in the presence of competitive antagonist whereas it is not modified in a significant manner in the presence of 10 and 50 μM of 805.

FIG. 9C represents the inhibition of the maximum response of cAMP induced by the NKA in the presence of an increasing concentration of 805. The x-axis represents the logarithm of the 805 concentration and the y-axis the maximum value of the production of cyclic AMP. The inhibition constant KI is of the order of 6 μM.

FIGS. 10A, 10B, 10C, 10D and 10E illustrate the effect of 805 on the calcium responses associated with the receptor EGFP-NK2R in the HEK 293 cells.

FIGS. 10A, 10B and 10C show the fluorescence of the INDO-1 at 400 m as a function of time in seconds.

The responses are recorded using the calcium-sensitive fluorescent probe INDO-1. For each concentration of agonist NKA (1 nM, 5 nM and 1 μM) a control response (white circle) and a response in the presence of 50 μM of 805 (black circle) are measured (FIGS. 10A, 10B and 10C). In all cases, an increase is noted in the duration of the calcium response in the presence of 805, without significant effect on the amplitude or on the kinetics of establishment of the response.

FIG. 10D indicates the variations in calcium responses obtained for a concentration of fixed agonist (5 nM of NKA) and variable concentrations (10 and 50 μM) of 805. The standardized calcium concentration is represented as a function of time in seconds. The curve with the white circles corresponds to a measurement carried out in the presence of 5 nM of NKA and without 805; the curve with the black circles corresponds to a measurement carried out in the presence of 5 nM of NKA and 10 μM of 805; and the curve with the black squares corresponds to a measurement carried out in the presence of 5 nM of NKA and 50 μM of 805.

In all cases an increase is noted in the duration of the calcium response in the presence of 805.

FIG. 10E show a quantification of the half-return time (time required for the calcium concentration to be half of its maximum value) at the initial value of the calcium response recorded in the presence of 0, 10 and 50 μM of 805.

FIGS. 11A, 11B, 11C, 11D, 11E, 11F and 11G illustrate the effect of 805 on the binding of fluorescent truncated neurokinin (NKA4-10 TR7) marked with the Texas red fluorescent group and the associated calcium response.

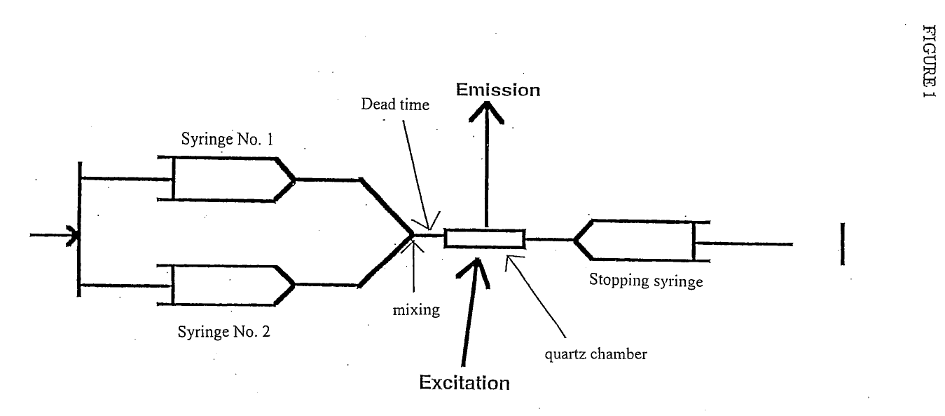

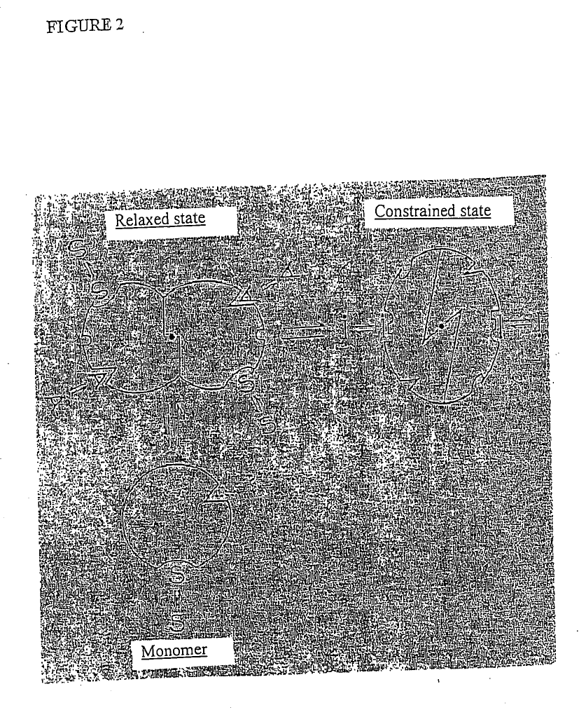

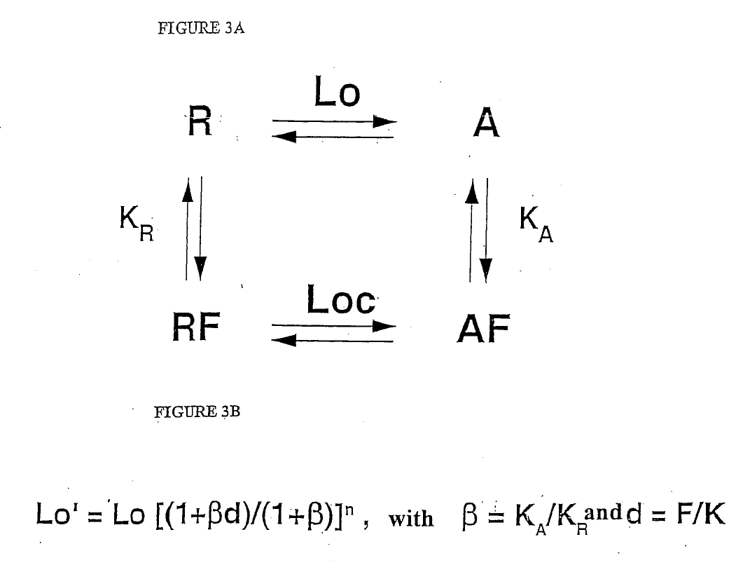

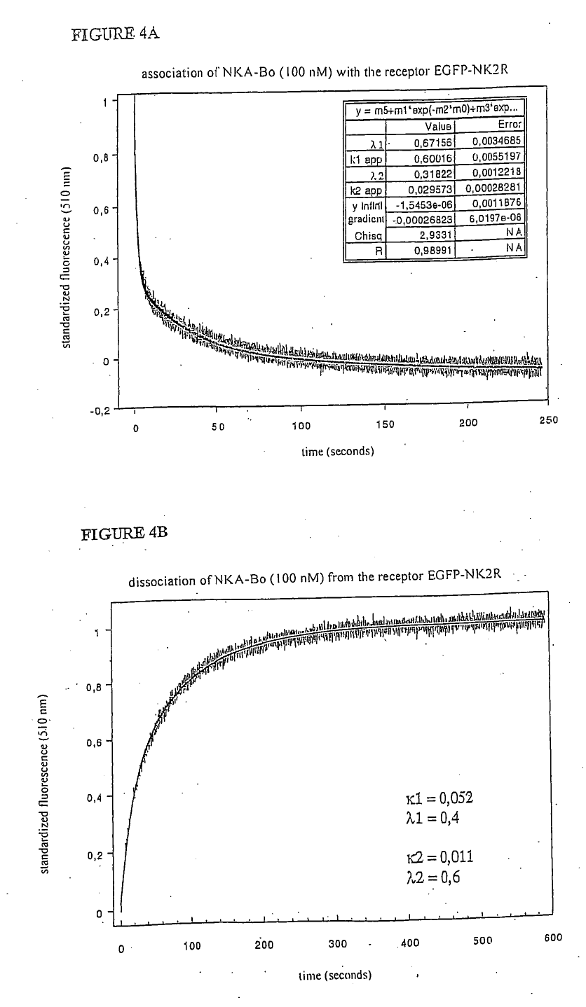

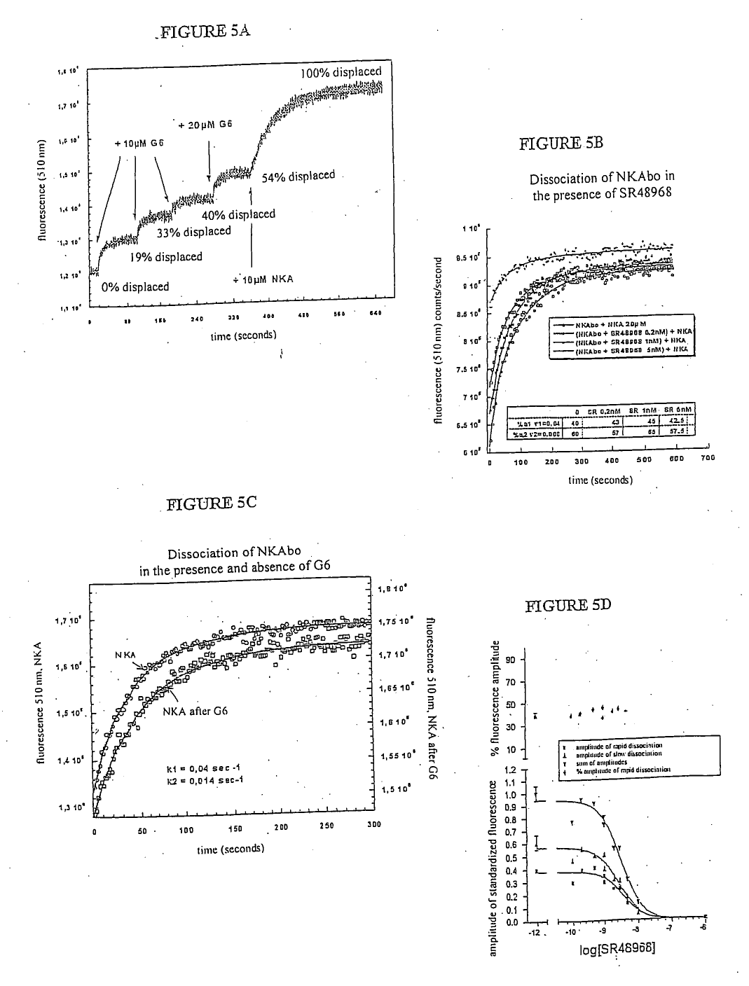

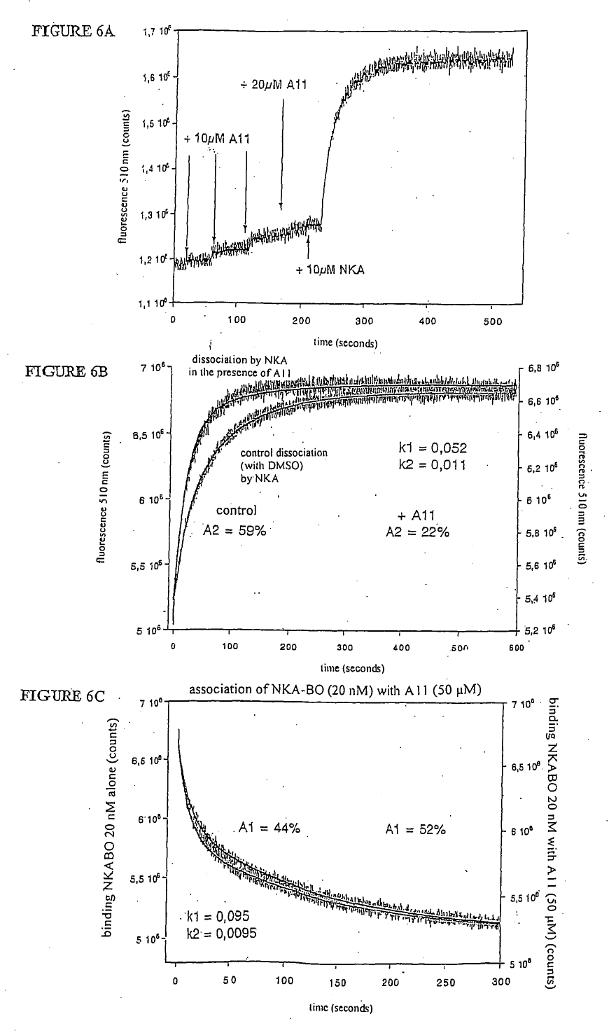

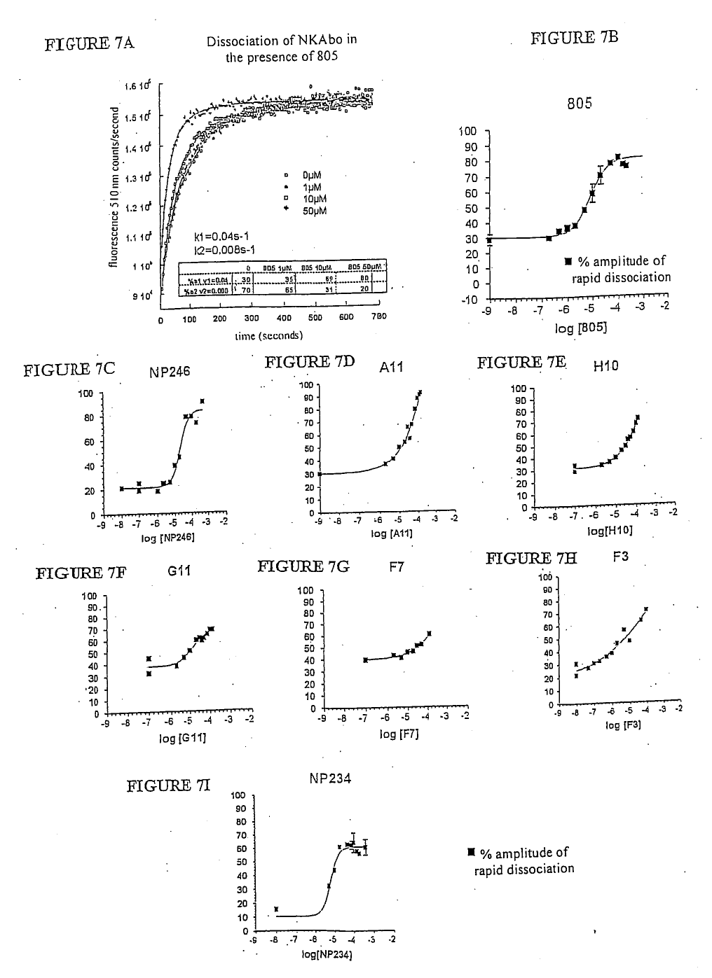

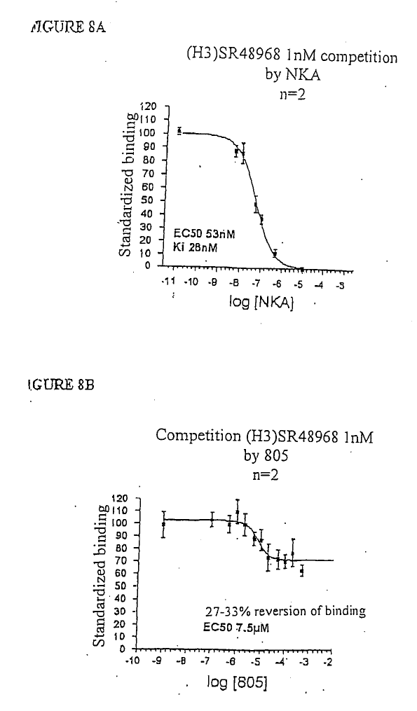

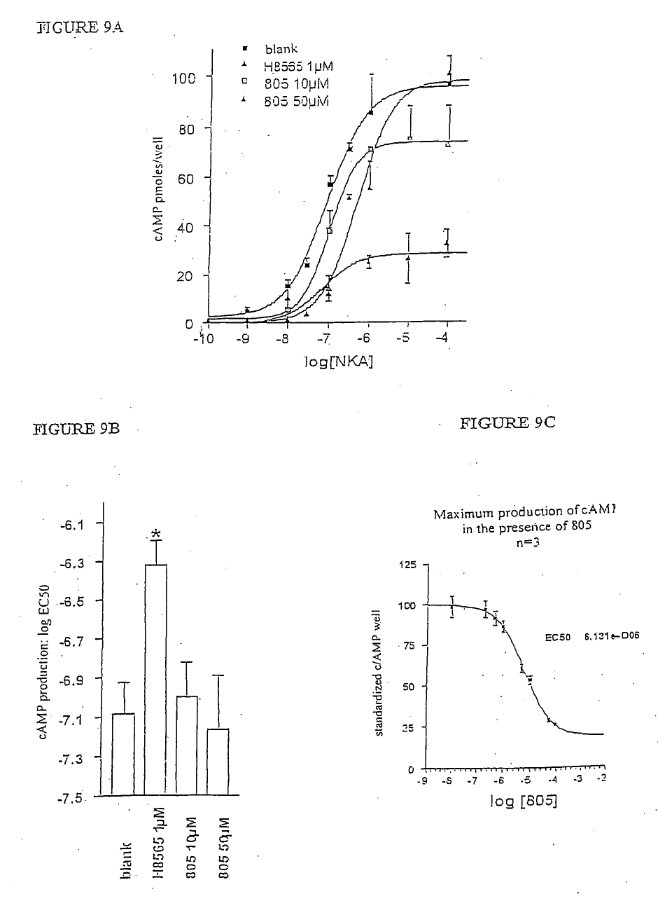

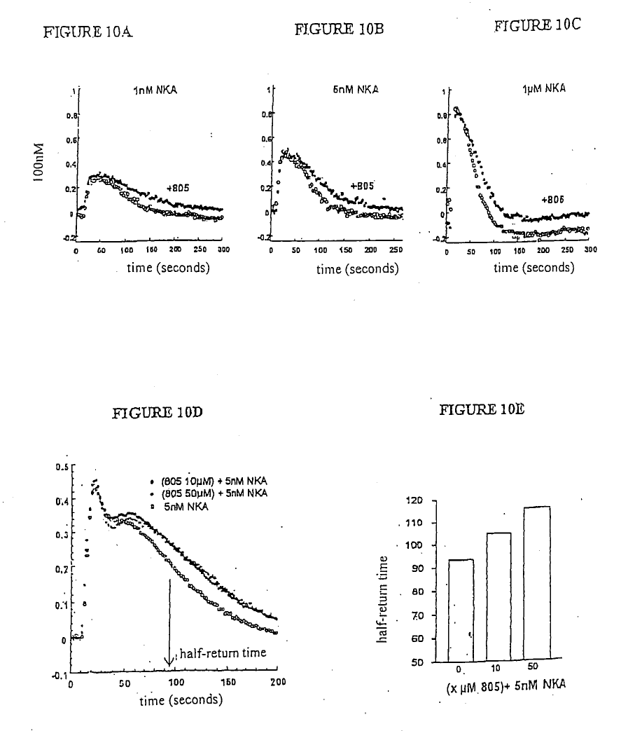

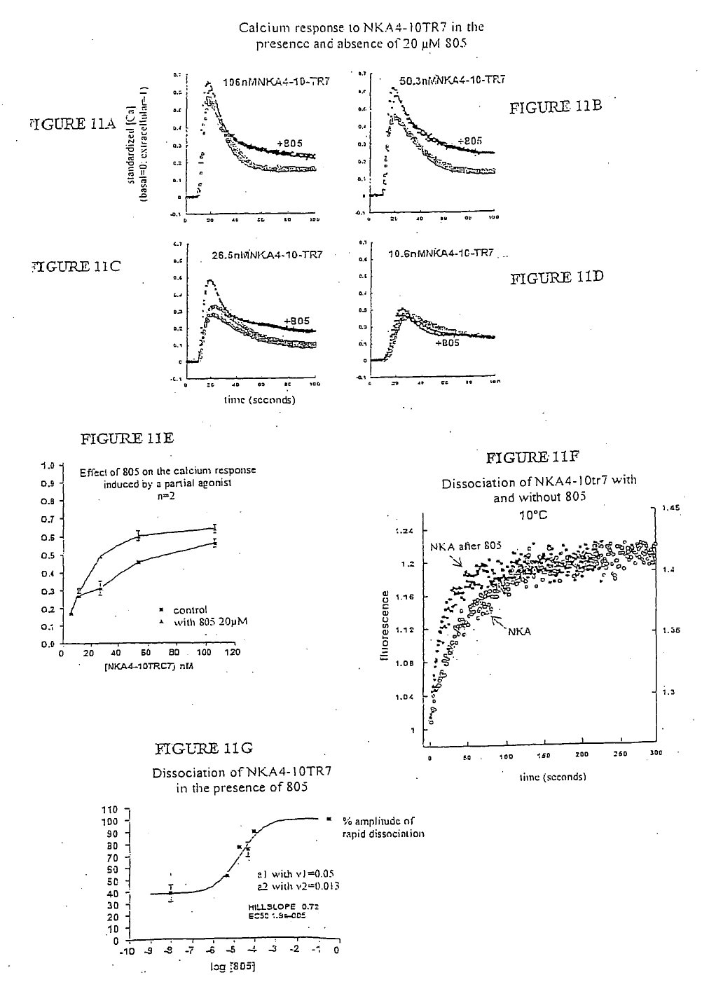

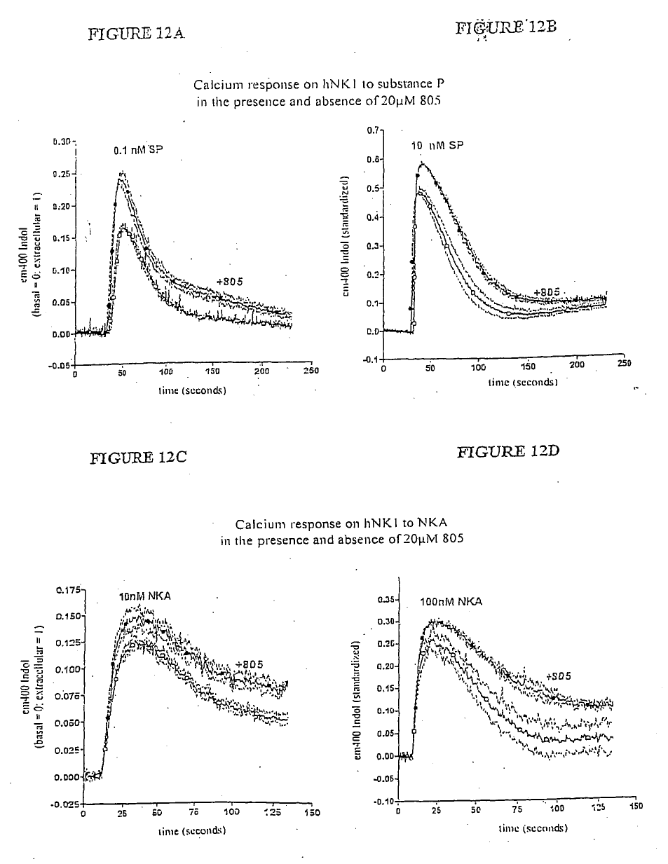

In FIGS. 11A to 11D, the calcium responses to 106 (FIG. 11A), 50.3 (FIG. 11B), 26.5 (FIG. 1C) and 10.6 nM (FIG. 11D) of NKA4-10 TR7 (NKA4-10 marked in position 7 by Texas Red) are recorded in duplicate, in the absence (curves with the white circles) and in the presence of 20 μM of 805 (curves with the black circles). The standardized calcium concentration is represented as a function of time in seconds. It is noted that 805 causes an increase in the amplitude of the calcium response, and a persistence of the signal.

FIG. 11E represents the relationship between the amplitude of the calcium response and the concentration of agonist. The amplitude of the standardized calcium peak is represented relative to the concentration of extracellular calcium, as a function of the concentration of NKA4-10 TR7. The curve with the black squares corresponds to the control measurement (without 805) and the curve with the triangles corresponds to the measurement carried out in the presence of 20 μM of 805.

FIG. 11F represents the dissociation of the NKA 4-10 TR7 bond. The x-axis represents time in seconds and the y-axis represents fluorescence. The complex formed between the receptor EGFP-NK2R and the NKA4-10 TR7 (106 nM) is reversed (at 10° C.) by the non-fluorescent NKA (20 μM) in the absence and in the presence of 10 μM of 805. Analysis of the dissociation kinetics shows two monoexponential relaxations the rate constants of which (0.05 sec⁻¹ and 0.013 sec⁻¹) are identical in the presence and in the absence of 805, but the relative amplitudes of which change such that the slow dissociation represents 42% of the total signal in the control and 12% of the total signal in the presence of 805.

FIG. 11G shows the percentage of the amplitude of the rapid dissociation of NKA 4-10 TR7 as a function of the logarithm of the 805 concentration. The amplitude of the rapid dissociation of NKA 4-10 TR7 is measured in the presence of variable concentrations of 805. The rapid dissociation represents approximately 40% of the total dissociation in the absence of 805 and up to 90% of the amplitude of dissociation at 100 μM. The apparent affinity for the variation in amplitude is of the order of 20 μM.

FIGS. 12A, 12B, 12C and 12D represent the calcium responses evoked by the substance P (endogenous ligand) on the human receptor NK1 (FIGS. 12A and 12B) and by the neurokinin A on the human receptor NK2 (FIGS. 12C and 12D), in the presence and in the absence of 805.

In FIGS. 12A and 12B, a suspension (1×10⁶ cells/ml) of HEK 293 cells expressing the wild-type human NK1 receptor, and charged with indo-1 (3 μM) is stimulated by 0.1 nM (FIG. 12A) or 10 nM (FIG. 12B) of substance P(SP) in the presence (black circles) and in the absence (white circles) of 805 molecules (20 μM).

In FIGS. 12C and 12D, a suspension (1×10⁶ cells/ml) of HEK 293 cells expressing the wild-type human NK2 receptor, and charged with indo-1 (3 μM) is stimulated by 10 nM (FIG. 12C) or 100 nM (FIG. 12D) of neurokinin A (NKA) in the presence (black circles) and in the absence (white circles) of 805 molecules (20 μM).

In all cases, it is noted that the 805 molecule causes an increase in the amplitude and duration of the calcium response caused by each agonist.

### EXAMPLES

### Example 1

### Measurement of the Association and Dissociation Rate of Neurokinin A Marked by the Fluorophore Bodipy with the Receptor NK2 of the Tachykinins Marked with EGFP, by Fluorescence Energy Transfer

Association (FIG. 4A): HEK 293 cells expressing the receptor EGFP-NK2R (NK2R marked with GFP) are put into suspension at a concentration of 2,000,000 cells/ml in Hepes physiological buffer (in mM: 137.5 NaCl; 1.25 MgCl₂; 1.25 CaCl₂; 6 KCl; 5.6 glucose; 10 Hepes; 0.4 NaH₂PO₄; 1% BSA (w/v); pH 7.4) and arranged in one of the reservoirs of the rapid mixing device. A solution of NKA-BO (neurokinin A marked with Bodipy) (200 nM) in the same buffer is arranged in the other reservoir of the rapid mixing device. The observation chamber is arranged in a SPEX fluorolog 3 spectrofluorimeter. The temperature of the reservoir of the syringes as well as of the mixing and observation chamber is fixed at 21° C. The excitation wavelength of the observation chamber is fixed at 470 nm, and the fluorescence emission wavelength is fixed at 510 nm. The sampling frequency of the experimental points is fixed at 50 Hz (1 point every 20 msec). The content of the reservoirs is driven towards the mixing and observation chamber using a pneumatic device allowing a flow rate of 4 ml/sec and the evolution of the mixture is recorded continuously from the moment of stopping of the thrust of the pistons of the reservoirs. The reaction of association of NKA-Bo with the receptor EGFP-NK2R is detected in the form of a reduction in the intensity of fluorescence of the GFP carried by the receptor NK2R. The experimental trace is adjusted by a multiexponential curve of type y=λ₁ exp (−k1ₐₚₚ×T)+λ₂ exp (−k2ₐₚₚ×T)+“straight”, where λ₁ and λ₂ are the amplitudes of the rapid and slow relaxations, k1ₐₚₚ and k2ₐₚₚ are the apparent rate constants of rapid and slow association, T is time and “straight” is a mathematical correction of the signal drift. The values of λ₁, λ₂, k1ₐₚₚ, k2ₐₚₚ for a final concentration of NKA-BO of 100 nM are respectively 67%; 32%; 0.6 sec⁻¹ and 0.03 sec⁻¹. The correlation coefficient of the adjustment of the experimental curve is R=0.989.

Dissociation (FIG. 4B): HEK 293 cells expressing the receptor EGFP-NK2R are put into suspension at a concentration of 1,000,000 cells/ml in Hepes physiological buffer (in mM: 137.5 NaCl; 1.25 MgCl₂; 1.25 CaCl₂; 6 KCl; 5.6 glucose; 10 Hepes; 0.4 NaH₂PO₄; 1% BSA (w/v); pH 7.4) and arranged in a 1 ml cuvette equipped with a magnetic stirrer on the cuvette holder of a SPEX fluorolog 3 spectrofluorimeter. 20 nM (final concentration) of NKA-Bo is added and the combination left to reach equilibrium for 10 minutes. The measurement of the dissociation kinetics of NKA-Bo is initiated by the manual addition of 10 μM (final concentration) of the competitive antagonist SR 48968 and recorded at 510 nm (excitation: 470 nm) at a rate of 1 point every 100 msec. The adjustment of the experimental curve is obtained using a sum of two exponentials having for rate constants k₁=0.052 sec⁻¹ and k₂=0.011 sec⁻¹ and for amplitudes λ₁=40% and λ₂=58%, respectively.

### Example 2

### Identification of the G6 Molecule Behaving as a Competitive Ligand, by Fluorescence Energy Transfer Between the NKA-Bo and the Receptor EGFP-NK2R

10⁶ cells HEK 293 expressing the receptor EGFP-NKR2 are incubated, in a millilitre of Hepes buffer, with 20 nm of NKA-Bo for 15 minutes. The mixture is then placed in a spectrofluorimetry cuvette and the reversion of the interaction between EGFP-NK2R and NKA-Bo is evaluated after the addition of G6 molecule (see Table 1) in increasing concentration (FIG. 5A). The quantification of the fraction of reversed complex is then carried out after the addition of a saturating quantity of NKA (10 μM) which gives access to the value of 100% reversion of the binding.

The comparison of the kinetics of dissociation of NKA-Bo by the NKA, in the presence and in the absence of G6 molecule (50 μM), shows that the G6 molecule does not alter the dissociation rate of NKA-Bo (FIG. 5B). The two experimental curves are correctly adjusted using two exponentials with a rapid rate constant k₁=0.04 sec⁻¹ and a slow rate constant k₂=0.01 sec⁻¹.

The control experiment shown in FIG. 5C indicates that a known molecule, competitive for the interaction of the NKA-Bo with its site, SR48968, causes a dissociation of NKA-Bo with kinetics identical to those recorded when the NKA-Bo is displaced by the NKA. At the different tested concentrations of SR 48968, it is noted that the dissociation of NKA-Bo occurs according to a biphasic process including a stage of rapid (k₁=0.04 sec⁻¹) and slow (k₂=0.008 sec⁻¹) dissociation. The relative amplitude of each stage remains constant for each concentration of SR 48968, as attested by the ratios of rapid/slow amplitudes close to 45/55.

Quantitative analysis of the dissociation of NKA-Bo in the presence of a range of increasing concentrations of SR 48968 reveals that for each concentration of SR 48968, the dissociation is produced not only according to a biphasic process with two rate constants (see FIG. 5C) but moreover that the amplitude ratio of the rapid dissociation and the slow dissociation remains constant (FIG. 5D). The slow dissociation always represents approximately 43% of the “rapid dissociation+slow dissociation” signal.

### Example 3

### Identification of a Molecule Behaving as an Allosteric Effector of the NK2 Receptor of Tachykinins

10⁶ HEK 293 cells expressing the receptor EGFP-NKR2 are incubated, in a millilitre of Hepes buffer, with 20 nM of NKA-Bo for 15 minutes. The mixture is then placed in a spectrofluorimetry cuvette and the reversion of the interaction between EGFP-NK2R and NKA-Bo is evaluated after the addition of A11 molecule in increasing concentration (FIG. 6A). Estimation of the fraction of reversed complex is then measured after the addition of a saturating quantity of NKA (10 μM). It is seen that the A11 molecule displaces only a small fraction (29%) of the NKA-Bo bound to its site. In contrast, it is noted that during the dissociation of NKA-Bo by the non-fluorescent NKA, the presence of A11 has the effect of accelerating the dissociation rate of NKA-Bo. This phenomenon is illustrated in FIG. 6B where the dissociation kinetics of NKA-BO by the NKA is measured in the presence and in the absence of A11 molecule. The two dissociation kinetics are better described by a sum of two exponential relaxations the rates of which are respectively k₁=0.052 sec⁻¹ for the rapid stage and k₂=0.011 sec⁻¹ for the slow stage. Given the association rate of NKA-Bo with its receptor (kₒₙ=5×10⁶ M⁻¹ sec⁻¹) it is deduced that the NKA-Bo is linked to two populations of sites the dissociation constants (K_{D}=k_{off}/kₒₙ) of which are respectively 10 nM (rapid dissociation—0.011 s⁻¹) and 2 nM (slow dissociation—0.052 s⁻¹). The amplitude of each relaxation is then given by the adjustment of the experimental traces. For the control dissociation (without A11), the rapid relaxation represents 41% and the slow 59%. After the addition of A11 (50 μM), the rapid relaxation represents 78% of the signal and the slow 22%. The A11 molecule therefore has the effect of stabilizing a fraction of the receptors in a state of lower affinity than that in which the NKA-Bo alone stabilizes it. The A11 molecule itself binds in a preferential manner to the conformation of the receptor which has a more modest affinity for the NKA-Bo. The binding of NKA-Bo is not inhibited by A11. It is produced on the same conformational states of the receptor, but the proportions of these two conformational states are modified by the presence of A11. As a result the dissociation kinetics of NKA-Bo is more rapid in the presence of A11 than in its absence. This acceleration of the dissociation kinetics of NKA-Bo does not result from a change in the intrinsic affinities of the conformations for NKA-Bo, but from a modification of the relative proportions of the states of high and low affinity.

Analysis of the effect of the A11 molecule on the association kinetics of NKA-Bo (FIG. 6C) reveals a modest but detectable acceleration. The rapid binding of NKA-Bo, recorded at a concentration of 20 nM using the rapid mixing equipment, is broken down into a sum of two exponentials the rates (kₐₚₚ) of which differ by a factor of 10. The rapid binding of NKA-Bo (kₐₚₚ=0.095 sec⁻¹) represents 44% of the total binding signal, in the absence of A11 (50 μM), whereas it represents 52% of the total signal in the presence of A11. The slow binding is carried out in both cases with an apparent rate (kₐₚₚ) equal to 0.0095 sec⁻¹, and an amplitude equal to 66% in the absence of A11, and 48% in the presence of A11. These results are interpreted according to a model described in Palanché et al. (2001) in the following terms:

the rapid binding like the slow binding is carried out in a manner which is controlled by the diffusion of NKA-Bo in the medium,

the apparent rate of the rapid binding increases in a linear manner with the concentration of NKA-Bo. It is therefore a binding controlled exclusively by the diffusion,

the apparent rate of the slow binding increases in a non-linear manner with the concentration of NKA-Bo and saturates at concentrations of ligand of the order of 500 nM. This slow kinetics reflects the slow interconversion of a conformational state of low affinity for NKA-BO towards a state of higher affinity the occupation of which is detected.

Thus, the receptor exists in at least two conformational states one of which pre-exists the addition of agonist and the other appears slowly over time in the presence of agonist.

The A11 molecule, by stabilizing the states of low affinity of the receptor, increases the proportion of rapid binding (of low affinity) to the detriment of slow binding which reflects the stabilization of a state of higher affinity

### Example 4

### Identification, by Analysis of the Binding of NKA-Bo, of Other Molecules Behaving as Allosteric Effectors of the Receptor NK2R of Tachykinins

The identification of other allosteric effectors of the receptor NK2R of tachykinins is carried out by analysis of the dissociation kinetics of NKA-Bo bound to the receptor.

10⁶ HEK 293 cells expressing the receptor EGFP-NK2R are put into suspension in Hepes buffer (see above) and incubated with NKA-Bo (20 nM) and one of the followings molecules: 805, NP246, A11, H10, G11, F7, F3 and NP 234. The A11 molecules and the known competitive antagonist SR 48968 are introduced into the experiment as positive controls. The dissociation of NKA-Bo is recorded, in a spectrofluorimeter cuvette equipped with a magnetic stirrer for 700 seconds, after adding 10 μM of non-fluorescent NKA to expel the bound NKA-Bo. The dissociation kinetics of NKA-Bo is recorded in the presence of increasing concentrations of each of the abovementioned molecules.

FIG. 7A shows a typical experiment for the measurement of the dissociation rate of NKA-Bo in the presence of increasing concentrations of the 805 molecule. These determinations make it possible to measure several experimental parameters: i) the dissociation rate of NKA-Bo which is then broken down into a sum of exponential relaxations and ii) the relative amplitude of each exponential relaxation which will produce the measurement of the fractional concentration of each of the conformational states detected in the experiment. It is thus determined that the dissociation of NKA-Bo occurs in two stages characterized by rate constants k₁=0.04 sec⁻¹ and k₂=0.008 sec⁻¹ and variable λ1/λ2 amplitudes: λ1/λ2=30/70 (without 805); λ1/λ2=35/65 (1 μM 805); λ1/λ2=9/31 (10 μM 805) and λ1/λ2=80/20 (50 μM 805).

FIGS. 7B to 7I show, for each molecule tested, the variation in amplitude of binding of NKA-Bo and the variation in amplitude of the rapid dissociation. These figures show that all the molecules indicated behave like allosteric effectors as they cause an increase in the amplitude of the rapid dissociation of NKA-Bo.

The increase in amplitude of the rapid dissociation reflects the ability of the molecules to stabilize a larger fraction of the receptors in the low-affinity state. It is thus seen that the initial fraction of receptors in the low-affinity state passes from approximately 25% in the absence of effector to 60-75% in the presence of the 805, NP234, A11, G11, F7, F3, and H10 molecules (at 100 μM of effector) and to almost 100% in the presence of A11 and NP246.

### Example 5

### Study of the Inhibition of the Binding of the Radioactive Competitive Antagonist SR 48968 by the Agonist NKA and the Allosteric Effector 805

25,000 cells expressing the receptor EGFP-NK2R are incubated for 3 hours at 4° C. in a final volume of 500 μl of Hepes buffer in the presence of 1 nM of [³H] SR48968 and variable concentrations of NKA or 805. On completion of the incubation, the reaction mixtures are filtered on GF/C (Whatmann) glass fibre filters previously incubated in Hepes buffer complemented by 1% (W/V) of powdered semi-skimmed milk, and rinsed three times with 5 ml of Hepes buffer at 4° C. The filters are then arranged in scintillation counter vials to which 3 ml of scintillating cocktail is added. The radioactivity is measured after 16 hours in a radioactivity counter. FIG. 8A shows that the agonist NKA quantitatively displaces the binding of SR 48968 with an apparent affinity Kᵢ=22 nM. On the other hand, the 805 effector is incapable of displacing the whole of the bound SR 48968, even when its concentration is of the order of the millimolar. It reverses only 27-33% of the binding of the radioligand with an apparent affinity Kᵢ=5 μM. The effect of 805 on the binding of SR 48968 does not reflect a competitive interaction but is rather of non-competitive nature.

### Example 6

### Quantification of the Effect of the 805 Molecule on the Accumulation Response of cAMP in HEK 293 Cells Expressing the Receptor EGFP-NK2R

30 mm 6-well cell-culture dishes are treated with collagen then seeded with 50,000 cells expressing the receptor EGFP-NK2R. After culture for two to three days, the medium is replaced by Hepes buffer containing phosphodiesterase IBMX (isobutyl-methyl-xhantine) inhibitor and incubated for 30 minutes with increasing concentrations of NKA in the presence or absence i) of competitive inhibitor H8565 (1 μM) or ii) of effector 805 (10 or 50 μM). The quantity of cAMP produced (expressed in pmoles/well) is determined according to a radioimmunological process described in Gicquiaux et al. (2002). This results in a measurement of the concentration-effect curve of NKA (FIG. 9A).

NKA alone is capable of stimulating the production of cAMP in cells expressing the receptor EGFP-NK2R with an EC₅₀ close to 100 nM.

In the presence of a fixed concentration of the competitive antagonist H8565, a reduction is noted in the apparent affinity of the NKA (EC₅₀=300 nM) without the maximum level of the response being affected (max. response=100%).

In the presence of the effector 805 at 10 or 50 μM, no variation is detected in the EC₅₀ of the NKA (EC₅₀=100 nM) but the maximum response never reaches the control value (Rₘₐₓ=0.7 for [805]=10 μM and Rₘₐₓ=0.3 for [805]=50 μM). The value of the concentration of 805 which leads to a 50% reduction in the maximum response of cAMP is of the order of 6 μM (FIG. 9C). Thus, the apparent affinity for the response, but not its maximum amplitude, is reduced in the presence of competitive antagonist, whereas the maximum amplitude of the response, but not its apparent affinity, is reduced by a negative allosteric effector.

### Example 7

### Quantification of the Effect of the 805 Molecule on the Calcium Accumulation Response Caused by the NKA-Bo in HEK 293 Cells Expressing the Receptor EGFP-NK2R

The calcium responses caused by the agonist NKA-Bo on cells expressing the receptor EGFP-NK2R are recorded after charging the cells with the calcium-sensitive fluorescent probe: indo-1, according to the protocol described in Vollmer et al. (1999). The cells are put into suspension at a rate of 500,000 cells/ml in Hepes buffer and arranged in a spectrofluorimeter cuvette. The excitation wavelength is fixed at 338 nm and the emission is recorded at 400 and 470 nm. The responses traced are the ratios of the signals 400/470. FIGS. 10A, 10B and 10C show that compound 805 (50 μM) increases the duration of the calcium response caused by 1, 5 and 1000 nM of NKA. In the same manner, compound 805, at concentrations of 10 and 50 μM, increases the duration of the calcium response caused by 5 nM of NKA (FIG. 10D).

Compound 805 therefore behaves as a positive allosteric effector of the calcium responses caused by the agonist NKA-Bo which increases the duration of the response. This is indicated in FIG. 10E where it can be seen that the time necessary to return to 50% of the maximum response passes from 91 seconds in the absence of 805 to 105 then 115 seconds when the concentration of 805 is 10 then 50 μM.

### Example 8

### Quantification of the Effect of the 805 Molecule on the Calcium Accumulation Response Caused by NKA 4-10 TR7, in HEK 293 Cells Expressing the Receptor EGFP-NK2R

The calcium responses caused by the truncated agonist NKA4-10 TR7 on cells expressing the receptor EGFP-NK2R are recorded after charging the cells with the calcium-sensitive fluorescent probe: indo-1 according to the protocol described in Vollmer et al. (1999). The cells are put into suspension at a rate of 500,000 cells/ml in Hepes buffer and arranged in a spectrofluorimeter cuvette. The excitation wavelength is fixed at 355 nm and the emission is recorded at 375 and 405 nm. The responses traced are the ratios of the 375 nm/405 nm signals. FIGS. 11A to 11D show that compound 805 (20 μM) increases the amplitude of the response caused by NKA 4-10 TR7 (106, 50.3, 26.5 and 10.6 nM). The variation in amplitude by 805 is illustrated in panel B of FIG. 11 for concentrations of 5, 10, 25, 50 and 106 nM of NKA 4-10 TR7. Compound 805 proves to be a positive allosteric effector of the calcium response caused by NKA 4-10 TR7 which reduces the 50%-effective dose from approximately 20 nM to approximately 15 nM.

### Example 9

### Quantification of the Effect of the 805 Molecule on the Dissociation of NKA4-10 TR7 from the Receptor EGFP-NK2R

NKA 4-10 corresponds to a truncated form of NKA which is found in the fluids of tumours of the medium intestine. The fluorescent NKA 4-10 is modified in position 7 in order to introduce a cysteine which is then marked by the Texas Red fluorescent group. The fluorescent NKA 4-10 is, like the fluorescent NKA, an agonist of the receptor EGFP-NK2R, to the extent that it is incapable of causing cAMP accumulation responses (Palanché et al., 2001).

The binding of fluorescent NKA 4-10 occurs according to a monoexponential process with which the development of a calcium response in HEK 293 cells expressing the receptor EGFP-NK2R is associated (Palanché et al., 2001).

At 10° C., the dissociation of NKA 4-10 rendered fluorescent using the Texas red group introduced at position 7 of the peptide (NKA4-10 TR7) occurs according to a biphasic process characterized by rapid and slow rate constants equal to k₁=0.5 sec⁻¹ and k₂=0.015 sec⁻¹, and respective amplitudes of 58% for the rapid dissociation and 42% for the slow dissociation (FIG. 11F). In the presence of 10 μM of 805, an acceleration of the dissociation is observed characterized by a modification of the amplitudes of the rapid (88%) and slow (12%) dissociation stages. A relationship is noted between the increase in dissociation rate of NKA4-10 TR7 and the concentration of 805 (FIG. 11G). The concentration of 805 which causes a 50% increase in the amplitude of the rapid dissociation of NKA4-10 TR7 is of the order of 20 μM.

### Example 10

### Potentialization of the Calcium Responses of the Wild-Type Human Receptors NK1 and NK2 by the 805 Molecule

The calcium response caused by the following agonists: substance P (SP) and neurokinin A (NKA), on cells expressing the wild-type human receptor NK1 or wild-type human receptor NK2 are recorded after charging the cells with the calcium-sensitive fluorescent probe: indo-1 (3 μM) according to the protocol described in Vollmer et al. (1999). The cells are put into suspension at a rate of 1,000,000 cells/ml in Hepes buffer (op. cit.) and arranged in a spectrofluorimeter cuvette. The excitation wavelength is fixed at 338 nm and the emission is recorded at 400 nm. The responses traced are the signals recorded at 400 nm. FIGS. 12A and 12B show that compound 805 (20 μM) increases the amplitude of the response caused by substance P (0.1 or 10 nM). FIGS. 12C and 12D show that compound 805 (20 μM) increases the amplitude of the response caused by neurokinin A (10 or 100 nM).

Compound 805 proves to be a positive allosteric effector of the calcium response caused by neurokinin A on the wild-type human receptor NK2 and of the response caused by substance P on the wild-type human receptor NK1.

### Example 11

### Identification of Allosteric Effectors of Muscarinic Receptor M1 of Acetylcholine

Fluorescent muscarinic receptor M1 is obtained by fusion of its cDNA with that of EGFP or YPFP as described in Ilien et al. (2003) and expressed in a stable manner in HEK 293 cells. The cells are put into suspension at a concentration of 1,000,000 cells/ml in Hepes physiological buffer (in mM: 137.5 NaCl; 1.25 MgCl₂; 1.25 CaCl₂; 6 KCl; 5.6 glucose; 10 Hepes; 0.4 NaH₂PO₄; 1% BSA (w/v); pH 7.4) and arranged in a 1 ml fluorescence cuvette, itself placed in the shaker rack of a spectrofluorimeter. The cell suspension is excited at 470 nm and the fluorescence emission is recorded at 510 nm (EGFP) or 530 nm (EYFP). The addition of 70 nM of bodipy (558-568)-pirenzepine causes a reduction in fluorescence emission of the fluorescent protein carried by the receptor which reflects the stage association of the ligand with the receptor. The time course of this stage is monitored by recording the emission at 510 or 530 nm. Alternatively, the complex between the receptor and the bodipy-pirenzepine is formed in a fluorescence cuvette placed in the spectrofluorimeter before adding an excess (5 μM) of atropine in order to initiate the dissociation stage of the receptor-ligand complex. The time course of this dissociation stage is recorded at 510 or 530 mm. In order to identify an allosteric effector of muscarinic receptor M1 of acetylcholine, the cells are pre-incubated with one or more, known or unknown molecule(s), and the time course of the association stage and/or dissociation stage is recorded. The kinetics recorded are then adjusted by a multiexponential curve of type y=λ₁ exp(−k1ₐₚₚ×T)+λ₂ exp(−k2ₐₚₚ×T)+“straight”, where λ₁ and λ₂ are the amplitudes of the rapid and slow relaxations, k1ₐₚₚ and k2ₐₚₚ are the apparent rate constants of rapid and slow association or dissociation, T is time and “straight” is a mathematical correction of the signal drift. An allosteric effector either accelerates, or slows down the time course either of the association stage, or of the dissociation stage, or both. This results in a relative variation in the rapid and slow association (or dissociation) amplitudes, in comparison to the control experimental condition (without effector).

### Example 12

### Identification of Allosteric Effectors of the Receptor CXCR4 of Chemokines

The fluorescent receptor CXCR4 is obtained by fusion of its cDNA with that of EGFP as described in Valenzuela et al. (2001) and in Palanché et al. (2001) and expressed in a stable manner in HEK 293 cells. The cells are put into suspension at a concentration of 1,000,000 cells/ml in Hepes physiological buffer (in mM: 137.5 NaCl; 1.25 MgCl₂; 1.25 CaCl₂; 6 KCl; 5.6 glucose; 10 Hepes; 0.4 NaH₂PO₄; 1% BSA (w/v); pH 7.4) and arranged in a 1-ml fluorescence cuvette itself placed in the shaker rack of a spectrofluorimeter. The suspension of cells is excited at 470 nm and the fluorescence emission is recorded at 510 nm. The addition of 50 nM of Texas Red-SDF1α causes a reduction in fluorescence emission of the fluorescent protein carried by the receptor which reflects the association stage of the ligand with the receptor. The time course of this stage is monitored by recording the emission at 510 nm. Alternatively, the complex between the receptor and the Texas Red-SDF1α is formed in a fluorescence cuvette placed in the spectrofluorimeter before adding an excess (500 nM) of SDF1α in order to initiate the dissociation stage of the receptor-ligand complex. The time course of this dissociation stage is registered at 510 nm. In order to identify an allosteric effector of the receptor CXCR4, the cells are pre-incubated with one or more known or unknown molecule(s), and the time course of the association stage and/or of dissociation stage is recorded. The kinetics recorded are then adjusted by a multiexponential curve of type y=λ₁ exp(−k1ₐₚₚ×T)+λ₂ exp(−k2ₐₚₚ×T)+“straight”, where λ₁ and λ₂ are the amplitudes of the rapid and slow relaxations, k1ₐₚₚ and k2ₐₚₚ are the apparent rate constants of rapid and slow association or dissociation, T is time and “straight” is a mathematical correction of the signal drift. An allosteric effector either accelerates, or slows down, the time course either of the association stage, or of the dissociation stage, or both. This results in a relative variation in the amplitudes of rapid and slow association (or dissociation), in comparison with the control experimental condition (without effector).

### REFERENCES

Alber and Kawasaki (1982) J. Mol. Appl. Gen., 1, 419-434,
Baulmann et al. (2000) Neuroscience, 95, 813-820,
Beaucage and Caruthers (1981) Tett. Lett., 22, 1859-1869,
Befort et al. (1996) Neurochem. Res., 11, 1301-1307,
Birdsall et al. (1999) Mol. Pharmacol., 55, 778-786,
Brisco, J. et al. (1996) Phylos. Trans. R. Soc. Lond. B. Biol. Sci., 351, 167-171,
Bruns & Ferguns (1990) Mol Pharmacol, 38, 939-949,
Burstein et al (1997) Mol. Pharmacol. 51, 312-319,
Chalfie (1995) Photochem. Photobiol., 62, 651-656,
Clegg (1995) Current Opinion in Biotechnol., 6, 103-110,
Cormack et al. (1995) Gene, 173, 33-38,
Cormack et al. (1996) Gene, 173, 1246-1251,
Crameri et al. (1996) Nature Biotechnol., 14, 315-319,
Ehrig et al. (1995) FEBS Lett. 367, 163-166
Fawzi et al. (2001) Mol. Pharmacol. 59, 30-37,
Förster (1951) in Fluoreszenz organischer Verbindung. Vandenhoek and Rupprecht, Göttingen,
Galzi & Alix (1997), Application international WO 98/55873,
Galzi et al. (1996) EMBO J., 15, 5824-5832,
Galzi et al. (1996) PNAS, 93, 1853-1858,
Gicquiaux et al. (2002) J. Biol. Chem. 277, 6645-6655,
Haas et al. (1996) Curr. Biol., 6, 315-323,
Hall (2000) Mol. Pharmacol., 58, 1412-1423,
Hausdorff et al. (1990) J. Biol. Chem 265, 1388-1393,
Heim & Tsien (1996) Current Biology, vol. No. 6, p 178-182,
Heim, Cubitt and Tsien (1995) Nature, 373, 663-664,
Hille, B. (1992) in Ion channels of excitable membranes, Sinauer Associates, Sunderlands, Massachussets,
Hitzeman et al. (1980) J. Biol. Chem., 255, 12073-12080,
Hoare & Strange (1995) Biochem. Soc. Trans., 23, 92S,
Hoare et al. (2000) British J. Pharmacol. 130, 1045-1059,
Honneger et al. (1988) EMBO J. 7, 3053-3060,
Ihle, J. N. (1995) Nature 377, 591-594,
Ilien et al. (2003) J. Neurochem 85, 768-778,
Jakubik et al. (1997) Mol. Pharmacol., 52, 172-179,
Ko et al. (2002) Mol. Cell. Biol., 22, 357-369,
Kostenis and Mohr (1996), Trends in Pharmacol. Sci., 17, 280-283,
Krause et al. (1997) Mol Pharmacol., 53, 283-294
Lakey et al. (1991) J. Mol. Biol., 218, 639-653
Lazareno & Birdsall (1995) Mol. Pharmacol., 48, 362-378
Lefkowitz (1998) J. Biol. Chem., 273, 18677-18680,
Mangelsdorf et al. (1995), Cell, 83, 835-839,
Massot et al. (1996), Mol. Pharmacol., 50, 752-762,
McKnight et al. (1985) EMBO J, 4, 2093-2099,
Miyawaki et al. (1997) Nature, vol. 388, p 882-887,
Monod et al. (1965) J. Mol. Biol., 12, 88-118,
Moutou et al. (1994) Tet. Lett., 35, 6883-6886,
Mulle et al. (1992) Neuron., 8, 937-945,
Nicolini et al. (1995) Biosens. Bioelection., 10, 723-733,
Nunnari et al. (1987) J. Biol. Chem., 262, 12387-12392,
Palanché et al. (2001) J. Biol. Chem., 276, 34853-34861,
Pan et al. (2001) Aneastesiology, 95, 416-420,
Prasher et al. (1992) Gene, 111, 229-233,
Reichel et al. (1996) Proc. Natl. Acad. Sci., 93, 5888-5893,
Rubin and Changeux (1966) J. Mol. Biol., 21, 265-274,
Russell (1985) Gene, 40.125-130,
Subramani et al. (1981) Mol Cell. Biol., 1, 854-864,
Tucek and Proska (1995) TIPS, 16, 205-212,
Valenzuela et al. (2001) J. Biol. Chem., 276, 26550-26558,
Vasuvedan et al. (1992) FEBS Lett., 311, 7-11,
Vlack et al. (1988) J. Gen. Virol., 69, 765-776,
Vollmer et al (1999) J. Biol. Chem., 274, 37915-37922,
Vuong and Chabre (1990) Nature, 346, 71-74,
Ward et al. (1980) Photochem. Photobiol., 31, 611-615,
Waugh et al. (1999) J. Pharm. Exp. Ther, 291, 1164-1171,
Weill et al. (1999) J. Neuro. Chem., 73, 791-801,
Wu and Brand (1994) Anal. Biochem., 218, 1-13,
Wurtz, J. L. et al. (1996) Nature Struct. Biol., 3, 206,
Yan et al. (1996) Nucleic Ac. Res., 24, 4592-4593,
Yarden, Y. and Ullrich, A. (1988) Biochemistry, 27, 3113-3119,
Young et al. (1982) in Genetic Engineering of microorganisms for chemicals (Hollaender et al. eds), Plenum Press, NY 1982,
Yuan et al. (2000) J. Biol. Chem., 275, 2157-2164,
Zolotukhin et al. (1996) J. Virol., 70, 4646-4654.

1. Process for detecting an allosteric effector of a receptor, by determination of the variation:
in the dissociation and/or association kinetics of the complex formed between the abovementioned receptor and one of its ligands in the presence of said allosteric effector, relative to the dissociation and/or association kinetics of the complex formed between said receptor and said ligand, in the absence of said effector,
and/or in the amplitude of the bond formed between the abovementioned receptor and one of its ligands in the presence of said allosteric effector, relative to the amplitude of the bond formed between said receptor and said ligand, in the absence of said effector,
provided that when the variation in the abovementioned amplitude is negative, the existence of the variation in the abovementioned kinetics is also detected,
said receptor and said ligand being involved in at least one biological response under appropriate physiological conditions, and the allosteric effector being capable of modulating at least one of the responses,
said receptor being marked by a fluorescent protein chosen from the fluorescent proteins obtained or derived from autofluorescent proteins of cnidaria, the molecular extinction coefficient of which is greater than approximately 14000 M⁻¹·cm⁻¹ and the fluorescence quantum efficiency is greater than approximately 0.38,
said ligand being marked by a marker constituted:
either by a molecule capable of absorbing the light emitted by the fluorescent protein,
or by a fluorescent substance,
said determinations of variation in dissociation and/or association kinetics and of variation in amplitude being carried out by fluorescence energy transfer:
between the abovementioned fluorescent protein and the abovementioned fluorescent substance, the fluorescent substance being such that either it is excitable at the emission wavelength of the abovementioned fluorescent protein, or it emits at the excitation wavelength of the abovementioned fluorescent protein, or
between the abovementioned fluorescent protein and the abovementioned molecule capable of absorbing the light emitted by the fluorescent protein.

2. Process for detecting an allosteric effector of a receptor, by determination of the variation:
in the dissociation and/or association kinetics of the complex formed between the abovementioned receptor and one of its ligands in the presence of said allosteric effector, relative to the dissociation and/or association kinetics of the complex formed between said receptor and said ligand, in the absence of said effector,
and/or in the amplitude of the bond formed between the abovementioned receptor and one of its ligands in the presence of said allosteric effector, relative to the amplitude of the bond formed between said receptor and said ligand, in the absence of said effector, provided that when the variation in the abovementioned amplitude is negative, the existence of the variation in the abovementioned kinetics is also detected,
said receptor and said ligand being involved in at least one biological response under appropriate physiological conditions, and the allosteric effector being capable of modulating at least one of the responses,
said receptor being marked by a fluorescent protein chosen from:
green fluorescent protein (GFP or EGFP), cyan fluorescent protein (CFP or ECFP), yellow fluorescent protein (YFP or EYFP) or GFPUV, or,
variants derived from GFP, CFP, YFP or GFPUV, by addition, deletion or substitution of one or more amino acids, provided that these variants preserve the property of fluorescence,
or fragments of GFP, CFP, YFP or GFPUV, or fragments of the abovementioned variants, provided that these fragments preserve the property of fluorescence,
said ligand being marked by a marker constituted:
either by a molecule capable of absorbing the light emitted by the fluorescent protein,
or by a fluorescent substance,
said determinations of variation in dissociation and/or association kinetics and variation in amplitude being carried out by fluorescence energy transfer:
between the fluorescent protein as defined above and the abovementioned fluorescent substance, the fluorescent substance being such that either it is excitable at the emission wavelength of the fluorescent protein, or it emits at the excitation wavelength of the fluorescent protein, or
between the fluorescent protein as defined above and the abovementioned molecule capable of absorbing the light emitted by the fluorescent protein.

3. Process according to claim 1, characterized in that the variation:
in the dissociation kinetics of the complex formed between the abovementioned receptor and one of its ligands in the presence of said allosteric effector, relative to the dissociation kinetics of the complex formed between said receptor and said ligand, in the absence of said effector,
and/or in the amplitude of the bond formed between the abovementioned receptor and one of its ligands in the presence of said allosteric effector, relative to the amplitude of the bond formed between said receptor and said ligand, in the absence of said effector is determined.

4. Process according to claim 1, characterized in that only the dissociation kinetics of the complex formed between the abovementioned receptor and one of its ligands in the presence of said allosteric effector, relative to the dissociation kinetics of the complex formed between said receptor and said ligand, in the absence of said effector are determined.

5. Process according to claim 1, characterized in that only the amplitude of the bond formed between the abovementioned receptor and one of its ligands in the presence of said allosteric effector, relative to the amplitude of the bond formed between said receptor and said ligand, in the absence of said effector is determined.

6. Process according to claim 1, characterized in that the ligand is an antagonist.

7. Process according to claim 1, characterized in that the ligand is an agonist.

8. Process according to claim 1, by determination of:
the variation in the amplitude of the bond formed between the abovementioned receptor and one of its ligands in the presence of said allosteric effector, relative to the amplitude of the bond formed between said receptor and said ligand, in the absence of said effector,
and optionally the variation in the dissociation kinetics of the complex formed between the abovementioned receptor and one of its ligands in the presence of said allosteric effector, relative to the dissociation kinetics of the complex formed between said receptor and said ligand, in the absence of said effector.

9. Process according to claim 8, characterized in that only the variation in the amplitude of the bond formed between the abovementioned receptor and one of its ligands in the presence of said allosteric effector, relative to the amplitude of the bond formed between said receptor and said ligand, in the absence of said effector, when said variation is positive is determined.

10. Process according to claim 8, characterized in that:
the variation in the amplitude of the bond formed between the abovementioned receptor and one of its ligands in the presence of said allosteric effector, relative to the amplitude of the bond formed between said receptor and said ligand, in the absence of said effector is determined, and that said variation is negative, which requires the determination of:
the variation in the dissociation kinetics of the complex formed between the abovementioned receptor and one of its ligands in the presence of said allosteric effector, relative to the dissociation kinetics of the complex formed between said receptor and said ligand, in the absence of said effector.

11. Process according to claim 10, characterized in that said variation in dissociation kinetics is positive or negative, which implies that the compound tested is an allosteric effector.

12. Process according to claim 10, characterized in that said variation in dissociation kinetics is zero, which implies that the compound tested is a competitor.

13. Products corresponding to one of the following formulae: 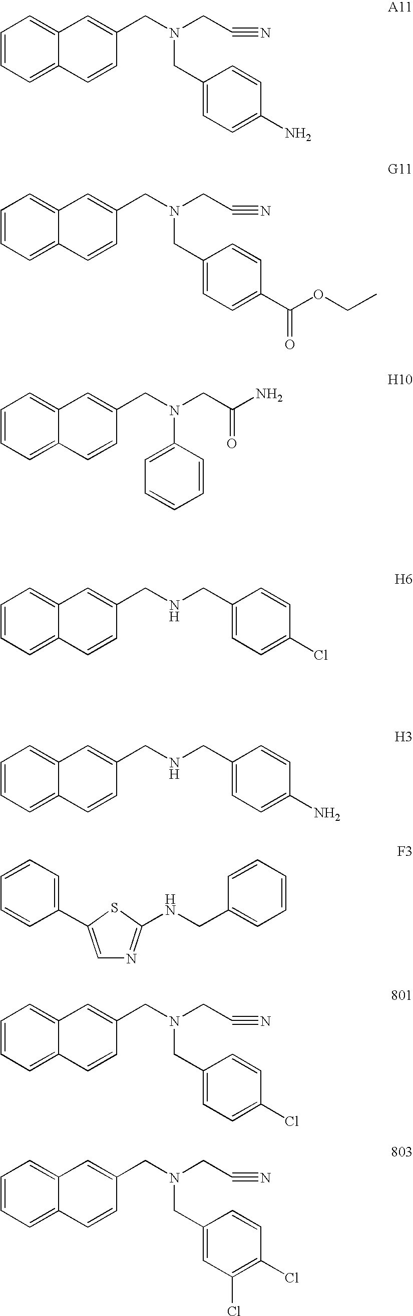 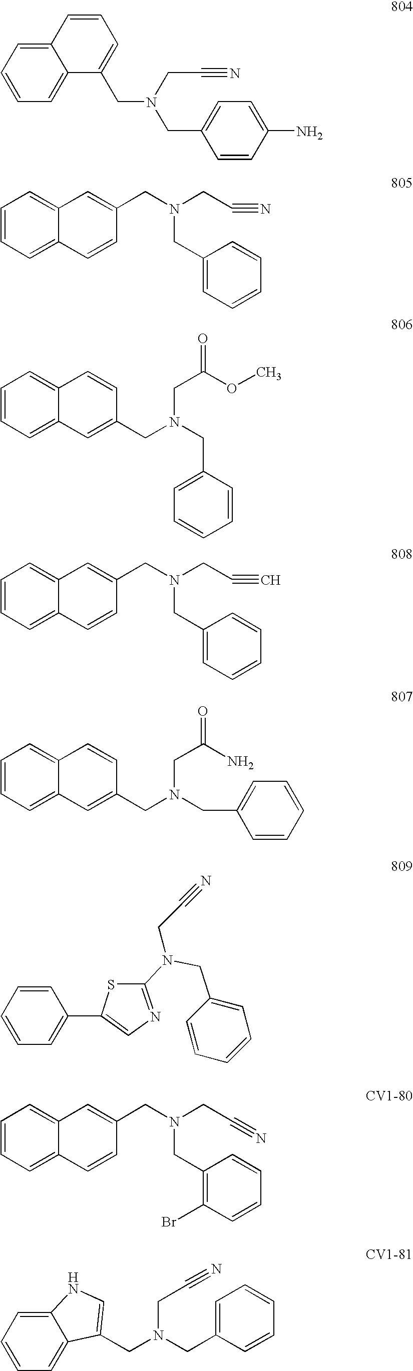 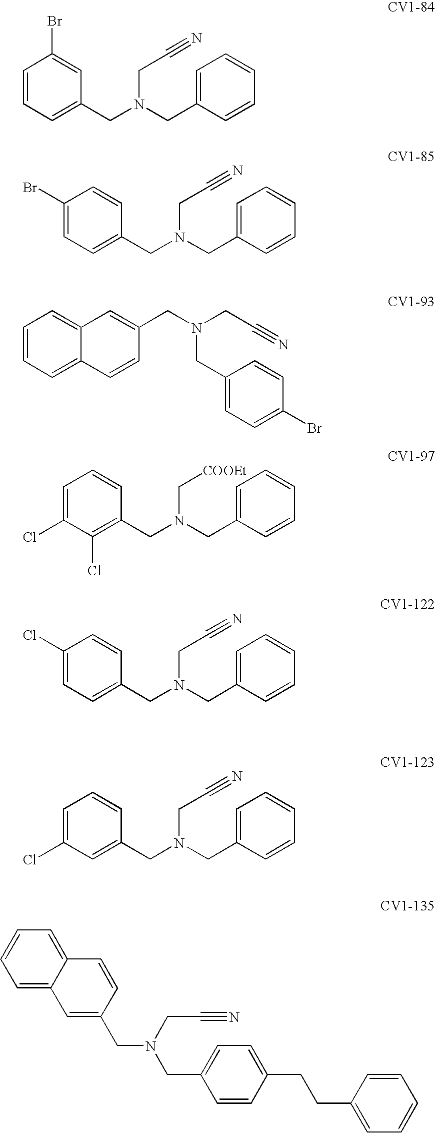
said products being compounds as detected by the process according to claim 1.
